SEQUENCE LISTING (15 sequences)

SEQ ID NO: 1 - DNA, 20 bp, Artificial
  An artificially synthesized primer for PCR
ttggcttgactcaggattta

SEQ ID NO: 2 - DNA, 20 bp, Artificial
  ′An artificially synthesized primer for PCR
atgctatcacctcccctgtg

SEQ ID NO: 3 - DNA, 22 bp, Artificial
  An artificially synthesized primer for PCR
ctcagctgggaaagctacagat

SEQ ID NO: 4 - DNA, 22 bp, Artificial
  An artificially synthesized primer for PCR
catgccaggtagttcttccatc

SEQ ID NO: 5 - DNA, 19 bp, Artificial
  An artificially synthesized target sequence for siRNA
cacagtatctcctagtcaa

SEQ ID NO: 6 - DNA, 19 bp, Artificial
  An artificially synthesized target sequence for siRNA
catgctgctcgagagaaac

SEQ ID NO: 7 - DNA, 19 bp, Artificial
  An artificially synthesized target sequence for siRNA
gttgctcaggatttggatt

SEQ ID NO: 8 - DNA, 19 bp, Artificial
  ′An artificially synthesized target sequence for siRNA
gcagctaatgctcctacca

SEQ ID NO: 9 - DNA, 19 bp, Artificial
  An artificially synthesized target sequence for siRNA
gaagcagcacgacttcttc

SEQ ID NO: 10 - DNA, 3189 bp, Homo sapiens
  CDS (464)..(1960)
gaactgtattcagcggcgacagcggcgactgcggcggccgcgggagggcatcccgttggggatccttccgcacactgaagagtacgtcttcgggtctacccctaatcacataatggctgtgtttaatcagaagtctgtctcggatatgattaaagagtttcgaaaaaattggcgtgctctttgtaactctgagagaactactctatgtggtgcagactccatgctcttggcattgcagctttctatggcggagaacaacaaacaggagagacggggtttcaccatgttagccaggatggtctcgatctcctgacttcgtgatccacccgcctcggcctcccaaagtgctaaaattacaggcgtgaaccaccaccacagtggagaatttacagtctctctcagtgatgttttattgacatggaaatacttgctccatgagaaattgaacttaccagttgaaaacatggacgtgactgaccattatgaggacgttaggaagatttatgatgatttcttgaagaacagtaatatgttagatctgattgatgtttatcaaaaatgtagggctttgacttctaattgtgaaaattataacacagtatctcctagtcaactactggattttctgtctggcaaacagtatgcagtaggtgatgaaactgatctttctataccaacatcaccaacaagtaaatacaaccgtgataatgaaaaggtgcagctgctagcaaggaaaattatcttttcatatttaaatctgctagtgaattcaaagaatgacctggctgtggcttatattctcaatattcctgatagaggactaggaagagaagccttcactgatttgaaacatgctgctcgagagaaacaaatgtctatctttttggtggccacgtcttttattagaacaatagagcttggagggaaaggatatgcaccaccaccatcagatcctttaaggacacatgtaaagggattgtctaattttattaatttcattgacaaattagatgagattcttggagaaataccaaacccaagcattgcagggggtcaaatactgtcagtgataaagatgcaactgattaaaggccaaaacagcagggatcctttttgcaaagcaatagaggaagttgctcaggatttggatttgaggattaaaaatattatcaattctcaagaaggtgttgtagctcttagcaccactgacatcagtcctgctcggccaaaatctcatgccataaaccatggtactgcatactgtggcagagatactgtgaaagccttattagttcttttggacgaagaagcagctaatgctcctaccaaaaacaaagcagagcttttatatgatgaggaaaacacaatccatcatcatggaacgtctattcttacactttttaggtctcccacacaggtgaataattcgataaaacccctaagagaacgcatctgtgtgtcaatgcaagagaaaaaaattaagatgaagcaaactttaattagatcccaatttgcttgtacttataaagatgactacatgataagcaaggataattggaataatgttaatttagcatcaaagcctttgtgtgttctttacatggaaaatgacctttctgagggtgtaaatccatctgttggaagatcaacaattggaacgagttttggaaatgttcatctggacagaagtaaaaatgaaaaagtatcaagaaaatcaaccagtcagacaggaaataaaagctcaaaaaggaaacaggtggatttggatggtgaaaatattctctgtgataatagaaatgaaccacctcaacataaaaatgctaaaatacctaagaaatcaaatgattcacagaatagattgtacggcaaactagctaaagtagcaaaaagtaataaatgtactgccaaggacaagttgatttctggccaggcaaagttaactcagttttttagactataaatttgtgtcttatatgctttaggtttatgtatctataaaccattcaccaaagacatgcttaatttttaagagatcaaggtgtaaattatgatgatttattattttggtctacagtgtatgtaaggttagtatgttaagcattgtttaaaaatactagtaagtcataattatgcagaattttcacaaagtttaatgcacagagaaagcatatcatttcagttactgatacatcttaacactactttcttttaaaacagacatttaacatacacaagttatagtagcagtatgggcttctcctcccattggcaattaaatgcttttattttcttctgaaaagatgatgtggaccaacaggtatcagacttgccaacaaggtcggtagactcttcccagcatacatctgagcactgaaggaagaagaaagtttaaattgtttaaaggactataattatcacacaaaatttattaagaaaaaaagaatggatctagtataactaattctgagtaaaccaaaatgataataattaattgttgctatttaatcccacatttttggcaggtgtaattgagccatggtcttatttgattttgttatgattgcatccaaattcactttaactcagagttctgtttaatggtggtaggatgtaagaattgaattttgaaaagactactcactgtcaaaatctctccttcctataggaaatttagctgagttttcttcatccccaatttctctcttttcttgtgttgattcagtattctgaactccattctcagctgggaaagctacagatccttttagtgcaagataaggttttatagccagattcagtggcagaccatgatttaagaaattatgtttggagcctgtgttctgtaaagagaaggttgatttggtttttagctatcgtattcggagtggaactataatacaattgtataatattcttgttgatcaattcaaagttactctgcactgtttttgactttttaaaaataccttagatgcaaatttataggagaaaaaacactttcagataagaggtgtttgctgggatggaagaactacctggcatgtaagaaatatcgtcagtcgtcctaatgcatattgtgactgtttgcatatacttctgtttataaaagtatcagttttacttttcagaggatttgtaagaatcatttaaattttcattgaaataaacgacaagtcacattgccactta

SEQ ID NO: 11 - protein, 498 aa, Homo sapiens
MDVTDHYEDVRKIYDDFLKNSNMLDLIDVYQKCRALTSNCENYNTVSPSQLLDFLSGKQYAVGDETDLSIPTSPTSKYNRDNEKVQLLARKIIFSYLNLLVNSKNDLAVAYILNIPDRGLGREAFTDLKHAAREKQMSIFLVATSFIRTIELGGKGYAPPPSDPLRTHVKGLSNFINFIDKLDEILGEIPNPSIAGGQILSVIKMQLIKGQNSRDPFCKAIEEVAQDLDLRIKNIINSQEGVVALSTTDISPARPKSHAINHGTAYCGRDTVKALLVLLDEEAANAPTKNKAELLYDEENTIHHHGTSILTLFRSPTQVNNSIKPLRERICVSMQEKKIKMKQTLIRSQFACTYKDDYMISKDNWNNVNLASKPLCVLYMENDLSEGVNPSVGRSTIGTSFGNVHLDRSKNEKVSRKSTSQTGNKSSKRKQVDLDGENILCDNRNEPPQHKNAKIPKKSNDSQNRLYGKLAKVAKSNKCTAKDKLISGQAKLTQFFRL

SEQ ID NO: 12 - DNA, 4001 bp, Homo sapiens
  CDS (172)..(3216)
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctctaaaaaaaaaaaaaaaaa

SEQ ID NO: 13 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 14 - DNA, 19 bp, Artificial
  An artificially synthesized target sequence for siRNA
cuagucaacuacuggauuu

SEQ ID NO: 15 - DNA, 19 bp, Artificial
  An artificially synthesized target sequence for siRNA
gauagagcgugaaggcgaa

(19) United States
(12) Patent Application Publication
(10) Pub. No.: US 2011/0263679 A1
(43) Pub. Date: Oct. 27, 2011

(54) C12ORF48 AS A TARGET GENE FOR CANCER THERAPY AND DIAGNOSIS

(75) Inventors: Yusuke Nakamura, Tokyo (JP); Hidewaki Nakagawa, Tokyo (JP); Akira Togashi, Kanagawa (JP)

(73) Assignee: Oncotherapy Science, Inc., Kanagawa (JP)

(21) Appl. No.: 13/061,098

(22) PCT Filed: Aug. 21, 2009
(86) PCT No.: PCT/JP2009/004020

§ 371 Date: Jun. 2, 2011

Related U.S. Application Data
(60) Provisional application No. 61/190,529, filed on Aug. 28, 2008.

(51) Int. Cl. A61K 31/713; G01N 33/574; C07H 21/00; C07K 16/18; A61P 35/00; C07H 21/04; C12N 15/85; G01N 33/573; C12Q 1/48; C12Q 1/68; C12Q 1/18

(52) U.S. Cl. 514/44 A; 435/6.14; 435/7.1; 536/24.31; 530/389.8; 436/501; 435/32; 435/6.13; 536/24.5; 435/320.1; 435/15

(57) ABSTRACT

Objective methods for diagnosing a predisposition to developing pancreatic cancer and prostate cancer, particularly pancreatic ductal adenocarcinoma (PDAC) and castration-resistant prostate cancer, are described herein. In one embodiment, the diagnostic method involves the step of determining an expression level of C12ORF48 using siRNAs targeting the C12ORF48 gene. The invention also features products such as siRNAs as well as to compositions containing them. The present invention further provides methods of screening for therapeutic agents useful in the treatment of C12ORF48 associated disease, such as a cancer, e.g. pancreatic cancer and prostate cancer, as well as methods of inhibiting the cell growth and treating or alleviating one or more disease symptoms. The invention also features products such as double stranded molecules, as well as vectors and compositions containing them.

### PRIORITY

The present application claims the benefit of U.S. Provisional Application No. 61/190,529, filed on Aug. 28, 2008, the entire contents of which are incorporated by reference herein.

### TECHNICAL FIELD

The present invention relates to methods of detecting and diagnosing a predisposition to developing cancer, particularly pancreatic cancer and prostate cancer, e.g., pancreatic ductal adenocarcinoma and castration-resistant prostate cancer. The present invention also relates to methods of screening for a candidate compound for treating and preventing a cancer associated with an over-expression of C12ORF48, particularly pancreatic cancer and prostate cancer, e.g., pancreatic ductal adenocarcinoma and castration-resistant prostate cancer. Moreover, the present invention relates to double-stranded molecules that reduce C12ORF48 gene expression and uses thereof. In particular, the present invention relates to C12ORF48.

### BACKGROUND ART

Pancreatic ductal adenocarcinoma (PDAC) is the fourth leading cause of cancer death in the western world and reveals the worst mortality among common malignancies, with a 5-year survival rate of only 5% (DiMagno E P, et al., Gastroenterogy 1999; 117:1464-84, Zervos E E, et al., Cancer Control 2004; 11:23-31). In 2007, it is estimated that 37,170 new cases of pancreatic cancer are diagnosed and a roughly equal number of deaths are attributed to pancreatic cancer in the United States (Jemal A, et al., CA Cancer J Clin 2007; 57:43-66). The majority of PDAC patients are diagnosed at an advanced stage, for which no effective therapy is available at present. Although only surgical resection offers a little possibility for cure, 80-90% of PDAC patients who undergo curative surgery die from their disseminated or metastatic diseases (DiMagno E P, et al., Gastroenterogy 1999; 117:1464-84, Zervos E E, et al., Cancer Control 2004; 11:23-31). Recent advances in surgery and chemotherapy including 5-FU or gemcitabine, with or without radiation, can improve patients' quality of life (DiMagno E P, et al., Gastroenterogy 1999; 117:1464-84, Zervos E E, et al., Cancer Control 2004; 11:23-31), but those treatments have a very limited effect on long-term survival of PDAC patients due to their extremely aggressive and chemoresistant nature. Hence, the management of most patients is focused on palliative measures (DiMagno E P, et al., Gastroenterogy 1999; 117:1464-84, Zervos E E, et al., Cancer Control 2004; 11:23-31).

On the other hand, prostate cancer (PC) is the most common malignancy in males and the second-leading cause of cancer-related death in the United States and Europe (Jemal A, et al., CA Cancer J Clin 2007; 57:43-66). The incidence of PC has been significantly increasing in most of developed countries due to prevalence of western-style diet and explosion of the aging population (Gronberg H, Lancet 2003; 361:859-64, Hsing A W, et al., Epidemiol Rev 2001; 23:3-13). The screening using serum prostatespecific antigen (PSA) lead to dramatic improvement of early detection of PC and resulted in an increase of the proportion of patients with a localized disease that could be curable by surgical and/or radiation therapies (Gronberg H, Lancet 2003; 361:859-64, Hsing A W, et al., Epidemiol Rev 2001; 23:3-13). However, 20-30% of these PC patients still suffer from the relapse of the disease (Scher H I, et al., J Clin Oncol 2006; 23:8253-61). Androgen/androgen receptor (AR) signaling pathway plays the central role in PC development and progression, and PC growth is usually androgen-dependent (Hsing A W, et al., Epidemiol Rev 2001; 23:3-13, Scher H I, et al., J Clin Oncol 2006; 23:8253-61). Hence, most of the patients with relapsed or advanced disease respond well to androgen-ablation therapy, which suppress testicular androgen production by surgical or medical castration. Nonetheless, they eventually acquire the tolerance to androgen-ablation therapy (castration) and more aggressive phenotype that are termed castration-resistant prostate cancers (CRPCs), for which there are very limited options such as doxotaxel plus predonisone (Tannock I F, et al., N Engl J Med 2004; 351:1502-12), but they can still offer the minimum effect on CRPCs. Hence, it is mostly demanded to identify molecular targets for CRPCs and develop novel therapies for CRPCs to target those molecules.

To overcome this dismal situation of both diseases, development of novel molecular therapies against good molecular targets is urgently needed. Toward this direction, detailed expression profiles of PDAC cells (Nakamura T, et al., Oncogene 2004; 23:2385-400) and CRPC cells (Tamura K, et al., Cancer Res 2007; 67:5117-25, WO2008/102906) were previously generated using genome-wide cDNA microarrays consisting of more than 30,000 genes, in combination with laser microbeam microdissection to enrich populations of cancer cells as much as possible.

### CITATION LIST

### Patent Literature

[PTL 1] WO2008/102906

### Non Patent Literature

[NPL 1] DiMagno E P, et al., Gastroenterogy 1999; 117:1464-84
[NPL 2] Zervos E E, et al., Cancer Control 2004; 11:23-31
[NPL 3] Jemal A, et al., CA Cancer J Clin 2007; 57:43-66
[NPL 4] Gronberg H, Lancet 2003; 361:859-64
[NPL 5] Hsing A W, et al., Epidemiol Rev 2001; 23:3-13
[NPL 6] Scher H I, et al., J Clin Oncol 2006; 23:8253-61
[NPL 7] Tannock I F, et al., N Engl J Med 2004; 351:1502-12
[NPL 8] Nakamura T, et al., Oncogene 2004; 23:2385-400
[NPL 9] Tamura K, et al., Cancer Res 2007; 67:5117-25

### SUMMARY OF INVENTION

The present invention relates to the discovery, through microarray analysis and RT-PCR, that C12ORF48 is over-expressed in several cancer cells. As demonstrated herein, functional knockdown of endogenous C12ORF48 by siRNA in cancer cell lines results in drastic suppression of cancer cell growth, suggesting its essential role in maintaining viability of cancer cells. Since it is only scarcely expressed in adult normal organs, C12ORF48 appears to be an appropriate and promising molecular target for a novel therapeutic approach with minimal adverse effect.

Accordingly, it is an object of the present invention to provide a method of diagnosing or determining a predisposition to cancer, particularly pancreatic ductal adenocarcinoma (PDAC) and castration-resistant prostate cancer (CRPC) in a subject by determining an expression level of C12ORF48 in a subject-derived biological sample, such as biopsy. An increase in the level of expression of C12ORF48 as compared to a normal control level indicates that the subject suffers from or is at risk of developing cancer, particularly pancreatic ductal adenocarcinoma (PDAC) and castration-resistant prostate cancer (CRPC). In the methods of the present invention, the C12ORF48 gene can be detected by appropriate probes or, alternatively, the C12ORF48 protein can be detected by anti-C12ORF48 antibody.

It is a further object of the present invention to provide methods for identifying an agent that inhibits the expression of the C12ORF48 gene or the activity of its gene product. Furthermore, the present invention provides methods for identifying a candidate agent for treating and/or preventing a C12ORF48 associated disease, such as cancer, e.g., pancreatic cancer and prostate cancer, particularly pancreatic ductal adenocarcinoma (PDAC) and castration-resistant prostate cancer (CRPC) or a candidate agent that inhibiting these cell growth. The methods of the present invention can be carried out in vitro or in vivo. A decrease in the expression level of the C12ORF48 gene and/or the biological activity of its gene product as compared to that in the absence of the test agent indicates that the test agent is an inhibitor of the C12ORF48 gene and thus may be used to inhibit the growth of a cell over-expressing the C12ORF48 gene, such as a cancerous cell, e.g., a pancreatic cancer cell or prostate cancer cell, particularly those of PDAC and CRPC. The candidate agent may be used to reduce a symptom of pancreatic cancer or prostate cancer, particularly pancreatic ductal adenocarcinoma (PDAC) or castration-resistant prostate cancer (CRPC).

It is yet a further object of the present invention to provide a method for inhibiting the growth of a cancerous cell over-expressing the C12ORF48 gene by administering agent that inhibits the expression of a C12ORF48 gene and/or a function of the C12ORF48 protein. Preferably, the agent is an inhibitory nucleic acid (e.g., an antisense, ribozyme, double stranded molecule). The agent may be a nucleic acid molecule or vector for providing double stranded molecule. Expression of the gene may be inhibited by introduction of a double stranded molecule into the target cell in an amount sufficient to inhibit expression of the C12ORF48 gene. The invention also provides methods for inhibiting the growth of a cancerous cell over-expressing the C12ORF48 gene in a subject, for example, in the context of therapeutic or preventative methods for the patients suffering from pancreatic cancer or prostate cancer, particularly pancreatic ductal adenocarcinoma (PDAC) or castration-resistant prostate cancer (CRPC).

It is yet a further object of the present invention to provide a pharmaceutical composition suitable for the treatment and/or prevention of pancreatic cancer or prostate cancer, particularly pancreatic ductal adenocarcinoma (PDAC) or castration-resistant prostate cancer, such a composition including a pharmaceutically acceptable carrier and an active agent including one or more of the double stranded molecules of the present invention or vectors encoding them. The double stranded molecules of the present invention are capable of inhibiting the expression of the C12ORF48 gene and inhibiting the growth of a cancerous cell over-expressing the C12ORF48 gene when introduced into the cell. Examples of such molecules include those that target at the sequence corresponding to the position of 595-613 nucleotide (SEQ ID NO: 5), 1133-1151 nucleotide (SEQ ID NO: 7) or 1310-1328 (SEQ ID NO: 8) nucleotide of SEQ ID NO: 10. Such molecules of the present invention include a sense strand and an antisense strand, wherein the sense strand includes a sequence including the target sequence, and wherein the antisense strand includes a sequence which is complementary to the sense strand. The sense and the antisense strands of the molecule hybridize to each other to form a double-stranded molecule.

It is yet a further object of the present invention to provide a detecting reagent and/or kit for diagnosing pancreatic cancer or prostate cancer, particularly pancreatic ductal adenocarcinoma (PDAC) or castration-resistant prostate cancer (CRPC), such a reagent or kit including an anti-C12ORF48 antibody.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

Various aspects and applications of the present invention will become apparent to the skilled artisan upon consideration of the brief description of the figures and the detailed description of the present invention and its preferred embodiments that follows:

FIG. 1 demonstrates the C12ORF48 over-expression in PDAC cells and CRPC cells. Part (A) depicts the results of semi-quantitative RT-PCR validating that C12ORF48 is over-expressed in the microdissected PDAC cells (lanes 1-9, left to right), as compared with normal pancreatic ductal cells which were also microdissected (“N.P.”), whole normal pancreatic tissue (“Panc.”), and vital organs (“heart”, “lung”, “liver”, and “kidney”). Expression of ACTB served as the quantitative control. Part (B) depicts the results of semi-quantitative RT-PCR validating that C12ORF48 is over-expressed in the microdissected CRPC cells (lanes 2-6, left to right), compared with normal prostate epithelial cells which were also microdissected (“NPro”), whole normal prostate tissue, brain and vital organs (“heart”, “lung”, “liver”, and “kidney”). Expression of ACTB served as the quantitative control. Part (C) depicts the results of Multiple Tissue Northern blot analysis for C12ORF48 expression, demonstrating that C12ORF48 showed expression in the testis, among the human adult organs. Part (D) depicts the results of Northern blot analysis for C12ORF48 expression, demonstrating that several PDAC cell lines (KLM-1, PK-59, PK-45P, and SUIT2) strongly expressed C12ORF48, while other normal adult organs did not express C12ORF48. Part (E) depicts the results of Northern blot analysis for C12ORF48 expression, showing that most of prostate cancer cell lines strongly expressed C12ORF48.

FIG. 2 demonstrates the effect of C12ORF48-siRNA on growth of cancer cells. Part (A) depicts the results of RT-PCR confirming the knockdown effect on C12ORF48 expression by si#595, si#1133, and si#1310, but not si#851 or a negative control siEGFP in PDAC cell line MiaPaCa2 (left) and PK-59 (right). ACTB was used to quantify RNAs. Part (B) depicts the results of MTT assays of each of MiaPaCa2 (left) and PK-59 (right) cells transfected with indicated siRNA-expressing vectors to C12ORF48 (si#595, si#1133, si#851, si#1310, or a negative control siEGFP). Each average is plotted with error bars indicating SD (standard deviation) after 6 days incubation with Geneticin. Y-axis means absorbance at 490 nm, and at 630 nm as reference, measured with a microplate reader. These experiments were carried out in triplicate. Part (C) depicts the results of colony formation assays of MiaPaCa2 (left) and PK-59 (right) cells transfected with each of indicated siRNA-expressing vectors to C12ORF48 (si#595, si#1133, si#851, si#1310, or a negative control siEGFP). Cells were visualized with 0.1% crystal violet staining after 2 weeks incubation with Geneticin.

FIG. 3 demonstrates that the C12ORF48 protein is localized in the nucleus. Part (A) depicts the results of Western blot analysis using anti-HA tagged antibody confirming that exogenous C12ORF48 is over-expressed in COS7 cells. Part (B) depicts the results of immunocytochemical analysis showing that exogenous C12ORF48 protein is localized in the nucleus. The green signal showed anti-HA stained C12ORF48 protein and DAPI staining (Blue) represented the nucleus in the cells. Part (C) depicts the results of Western blot analysis using anti-C12ORF48 antibody detecting endogenous C12ORF48 in PDAC cell lines. C12ORF48 was highly expressed in PDAC cell lines KLM1, SUIT2 and PK-1, while hardly detectable in non-cancerous cell lines (HEK-293, and COS7). beta-Actin served as the loading control. Part (D) depicts the results of immunohistochemical study using anti-C12ORF48 antibody. The strong positive staining of C12ORF48 was observed in the nucleus of PDAC cells (C1×40, C2×40). In normal pancreatic tissue, acinar cells and normal ductal epithelium cells showed no or little staining (N x40). In total, 33 of 62 (53%) PDAC tissues showed positive staining for C12ORF48.

FIG. 4 demonstrates that the Interaction of C12ORF48 with PARP1. Part (A) depicts the result of the silver staining of SDS-PAGE gel showing that 110 kDa band was differentially co-immunoprecipitaed with C12ORF48-Flag in HECK293 cells. LC-MS/MS analysis identified PARP1protein as a corresponding protein with this 110 kDa band. Part (B) depicts the results of Western blot analysis using anti-PARP1 antibody confirmed that PARP1 protein was co-immunoprecipitated with C12ORF48-Flag in HECK293 cells. Part (C) depicts the results of Flag-C12ORF48 expression vector and/or PARP1-Myc expression vector were co-transfected into HEK293 cells. Protein complexes containing Flag-C12ORF48 and/or PARP1-Myc were immunoprecipitated by c-Myc antibody. Western blotting using anti-Flag antibody indicated that Flag-C12ORF48 was co-immunoprecipitated with PARP1-Myc when both expression vectors were co-transfected.

FIG. 5 demonstrates that the induction of apoptosis by siRNA duplexes to knock down C12ORF48 or PARP1. Part (A) depicts the results of FACS analysis detecting an increasing proportion (65%) of subG1 populations in KLM-1 cells transfected with C12ORF48-siRNA, compared with cells transfected with control siRNA (27%). Part (B) depicts the results of detecting FACS analysis detected an increasing proportion (79%) of subG1 populations in KLM-1 cells transfected with PARP1-siRNA, compared with cells transfected with control siRNA (40%).

FIG. 6 demonstrates that C12ORF48 positively regulates PARP1 automodification in vitro. PARP1 automodification was measured by incorporation of [³²P] NAD+ and visualized by SDS-PAGE in the absence or in the presence of purified recombinant C12ORF48 protein. Lanel, purified recombinant His-tagged C12ORF48 protein alone; lane2, both His-tagged C12ORF48 and PARP1 protein; lane3, PARP1 protein alone. PARP1 automodification reflected by incorporation of [³²P] NAD+ was strongly enhanced in the presence of C12ORF48 protein, compared with in the absence of C12ORF48 protein.

FIG. 7 demonstrates that the C12ORF48 knockdown could reduce PARP1 activity in cancer cell extracts. Part (A) depicts the results of Western blot analysis using anti-C12ORF48 (upper) and annti-PARP1 (lower) antibodies confirming the knock-down effect of C12ORF48/PARP1 siRNA in KLM-1 cells. Part (B) depicts the results of the colorimetric PARP assay based on the incorporation of biotinylated ADP-ribose onto histone H1 proteins showing the PARP1 activities to poly(ADP-ribosyl)ate histone H1 in KLM-1 cell extracts transfected with C12ORF48-siRNA, or PARP1-siRNA were decreased 59.2%, and 55.5% respectively, compared with control-siRNA. Part (C) depicts the results of Western blot analysis using anti-C12ORF48 (upper) and annti-PARP1 (lower) antibodies confirming the knock-down effect of C12ORF48/PARP1 siRNA in SUIT-2 cells. Part (D) depicts the results of colorimetric PARP assay showing the PARP1 activities to poly(ADP-ribosyl)ate histone H1 in SUIT2 cell extracts transfected with C12ORF48-siRNA, or PARP1-siRNA were decreased 65.2% and 47.1% respectively, compared with control-siRNA. Part (E) depicts that the products of PARP1 enzymatic reaction, indicated as PAR, were detected after PAGE on Western blots using anti-pADPr antibody. PARP1 activity was drastically decreased in the cell extracts in C12ORF48 or PARP1 knockdown.

FIG. 8 demonstrates that the Over-expression of C12ORF48 could enhance the activity of PARP1 in cell extracts. Part (A) depicts that the cells were collected respectively in 12, 24, and 48 hours after the transfection of C12ORF48-expression vector, and the expression of C12ORF48 protein were detected using anti-C12ORF48 polyclonal antibody. Part (B) depicts that the PARP1 activities in cell extracts were measured by using the colorimetric PARP assay. Concordantly with C12ORF48 expression, the PARP1 activity in HEK293 cell extracts were also enhanced (P<0.0001, vs mock transfection)

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

In case of conflict, the present specification, including definitions, will control. In addition, the materials, methods, and examples are illustrative only and not intended to be limiting.

### DEFINITION

The words “a”, “an”, and “the” as used herein mean “at least one” unless otherwise specifically indicated.

The terms “isolated” and “purified” when used herein in relation to a substance (e.g., polypeptide, antibody, polynucleotide, etc.) indicate that the substance is substantially free from at least one substance that may else be included in the natural source. Thus, an isolated or purified antibody refers to antibodies that are substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “oligonucleotide”, “nucleotides”, “nucleic acids”, and “nucleic acid molecules” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules may be composed of DNA, RNA or a combination thereof.

Unless otherwise defined, the terms “cancer” refers to cancers over-expressing the C12ORF48 gene. Examples of cancers over-expressing C12ORF48 include, but are not limited to, pancreatic cancer and prostate cancer, more particularly pancreatic ductal adenocarcinoma and castration-resistant prostate cancer.

The C12ORF48 Gene or C12ORF48 Protein

The invention is based in part on the discovery that the gene encoding C12ORF48 is over-expressed in pancreatic ductal adenocarcinoma (PDAC) as compared to non-cancerous tissue. The cDNA of C12ORF48 is 3,189 nucleotides in length. The nucleic acid and polypeptide sequences of C12ORF48 are shown in SEQ ID NOs: 10 and 11, respectively. Additional sequence data is also available via following accession numbers.

C12ORF48: NM_{—}017915

According to an aspect of the present invention, functional equivalents are also considered to be “C12ORF48 polypeptides”. Herein, a “functional equivalent” of a protein is a polypeptide that has a biological activity equivalent to the protein. Namely, any polypeptide that retains the biological ability of the C12ORF48 protein may be used as such a functional equivalent in the present invention. Such functional equivalents include those wherein one or more amino acids are substituted, deleted, added, or inserted to the natural occurring amino acid sequence of the C12ORF48 protein. Alternatively, the polypeptide may be composed an amino acid sequence having at least about 80% homology (also referred to as sequence identity) to the sequence of the respective protein, more preferably at least about 90% to 95% homology, even more preferably 96% to 99% homology. In other embodiments, the polypeptide can be encoded by a polynucleotide that hybridizes under stringent conditions to the naturally occurring nucleotide sequence of the C12ORF48 gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human C12ORF48 protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will vary in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the human C12ORF48 protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

In general, modification of one, two or more amino acids in a protein will not influence the function of the protein. In fact, mutated or modified proteins (i.e., peptides composed of an amino acid sequence in which one, two, or several amino acid residues have been modified through substitution, deletion, insertion and/or addition) have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention. Thus, in one embodiment, the peptides of the present invention may have an amino acid sequence wherein one, two or even more amino acids are added, inserted, deleted, and/or substituted in the C12ORF48 sequence.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 5 or 6 amino acids or less, and even more preferably 3 or 4 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified polypeptides are included in the present C12ORF48 protein. However, the present invention is not restricted thereto and the C12ORF48 protein includes non-conservative modifications, so long as at least one biological activity of the C12ORF48 protein is retained. Furthermore, the modified proteins do not exclude polymorphic variants, interspecies homologues, and those encoded by alleles of these proteins.

Moreover, the C12ORF48 gene of the present invention encompasses polynucleotides that encode such functional equivalents of the C12ORF48 protein. In addition to hybridization, a gene amplification method, for example, the polymerase chain reaction (PCR) method, can be utilized to isolate a polynucleotide encoding a polypeptide functionally equivalent to the C12ORF48 protein, using a primer synthesized based on the sequence information of the protein encoding DNA (SEQ ID NO: 10). Polynucleotides and polypeptides that are functionally equivalent to the human C12ORF48 gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher, even more preferably 96% to 99% or higher. The homology of a particular polynucleotide or polypeptide can be determined by following the algorithm in “Wilbur and Lipman, Proc Natl Acad Sci USA 80: 726-30 (1983)”.

A Method for Diagnosing Cancer

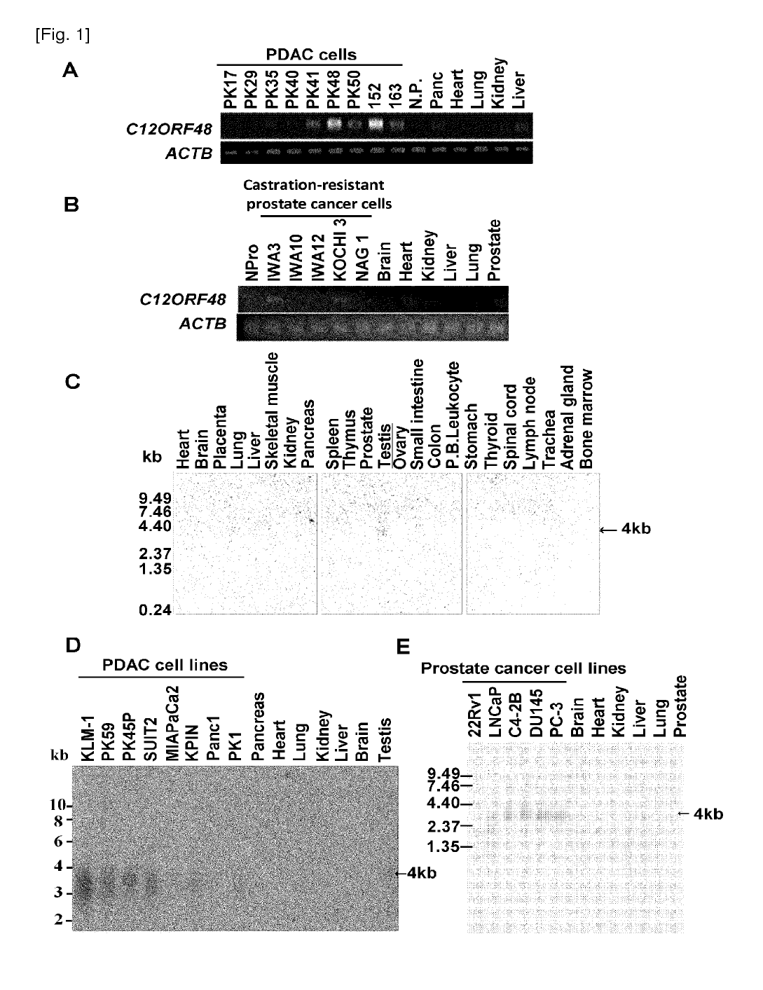

The expression of C12ORF48 was found to be specifically elevated in pancreatic cancer and prostate cancer, particularly pancreatic ductal adenocarcinoma (PDAC) and castration-resistant prostate cancer (CRPC) (FIG. 1). Accordingly, the C12ORF48 genes identified herein as well as their transcription and translation products find diagnostic utility as a marker for cancers such as pancreatic cancer and prostate cancer, particularly pancreatic ductal adenocarcinoma (PDAC) and castration-resistant prostate cancer (CRPC), and by measuring the expression of C12ORF48 in a sample, those cancers can be diagnosed. More particularly, the present invention provides a method for detecting, diagnosing and/or determining the presence of or a predisposition for developing cancer, more particularly PDAC or CRPC, by determining the expression level of C12ORF48 in the subject. In the context of the present invention, the term “cancer” indicates pancreatic cancer and prostate cancer that can be diagnosed by the present method include PDAC and CRPC.

According to the present invention, an intermediate result for examining the condition of a subject may be provided. Such intermediate result may be combined with additional information to assist a doctor, nurse, or other practitioner to determine that a subject suffers from the disease. That is, the present invention provides a diagnostic marker C12ORF48 for examining cancer.

Alternatively, the present invention provides a method for detecting or identifying cancer cells in a subject-derived pancreatic or prostate tissue sample, the method including the step of determining the expression level of the C12ORF48 gene in a subject-derived biological sample, wherein an increase in the expression level as compared to a normal control level of the gene indicates the presence or suspicion of cancer cells in the tissue.

Such result may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the C12ORF48 gene, plus a different aspect of the disease including tissue pathology, levels of known tumor marker(s) in blood, and clinical course of the subject, etc. For example, some well-known diagnostic pancreatic tumor markers in blood are BFP, CA19-9, CA72-4, CA125, CA130, CEA, DUPAN-2, IAP, KM0-1, NCC-ST-439, NSE, sICAM-1, SLX, Span-1, STN, TPA, YH-206 andelastase 1. Alternatively, diagnostic prostate tumor markers in blood such as BFP, IAP, alpha macroglobulin, PAP, PIPC, PSA gamma-Sm and TPA are also well known. Namely, in this particular embodiment of the present invention, the outcome of the gene expression analysis serves as an intermediate result for further diagnosis of a subject's disease state.

Of particular interest to the present invention are the following methods [1] to [10]:

[1] A method of detecting or diagnosing cancer in a subject, including determining an expression level of C12ORF48 in a subject-derived biological sample, wherein an increase of the level compared to a normal control level of the gene indicates that the subject suffers from or is at risk of developing cancer;

[2] The method of [1], wherein the expression level is at least 10% greater than the normal control level;

[3] The method of [1], wherein the expression level is detected by a method selected from among:

(a) detecting an mRNA including the sequence of C12ORF48,

(b) detecting a protein including the amino acid sequence of C12ORF48, and

(c) detecting a biological activity of a protein including the amino acid sequence of C12ORF48;

[4] The method of [1], wherein the cancer is pancreatic cancer or prostate cancer.

[5] The method of [4], wherein the pancreatic cancer is pancreatic ductal adenocarcinoma (PDAC);

[6] The method of [4], wherein prostate cancer is castration-resistant prostate cancer (CRPC);

[7] The method of [3], wherein the expression level is determined by detecting hybridization of a probe to a gene transcript of the gene;

[8] The method of [3], wherein the expression level is determined by detecting the binding of an antibody against the protein encoded by a gene as the expression level of the gene;

[9] The method of [1], wherein the biological sample includes biopsy, sputum or blood;

[10] The method of [1], wherein the subject-derived biological sample includes an epithelial cell;

[11] The method of [1], wherein the subject-derived biological sample includes a cancer cell; and

[12] The method of [1], wherein the subject-derived biological sample includes a cancerous epithelial cell.

The method of diagnosing cancer of the present invention will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of C12ORF48. The biological samples include, but are not limited to, bodily tissues which are desired for diagnosing or are suspicion of suffering from cancer, and fluids, such as biopsy, blood, sputum and urine. Preferably, the biological sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of C12ORF48 in the subject-derived biological sample is determined. The expression level can be determined at the transcription (nucleic acid) product level, using methods known in the art. For example, the mRNA of C12ORF48 may be quantified using probes by hybridization methods (e.g., Northern hybridization). The detection may be carried out on a chip or an array. The use of an array is preferable for detecting the expression level of a plurality of genes (e.g., various cancer specific genes) including C12ORF48. Those skilled in the art can prepare such probes utilizing the sequence information of C12ORF48. For example, the cDNA of C12ORF48 may be used as the probes. If necessary, the probe may be labeled with a suitable label, such as dyes, fluorescent and isotopes and the expression level of the gene may be detected as the intensity of the hybridized labels.

Furthermore, the transcription product of C12ORF48 may be quantified using primers by amplification-based detection methods (e.g., RT-PCR). Such primers can also be prepared based on the available sequence information of the gene. For example, the primers (SEQ ID NOs: 3 and 4) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of C12ORF48. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the diagnosis of the present invention. For example, the quantity of C12ORF48 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to C12ORF48 protein. Methods to prepare these kinds of antibodies for the detection of proteins are well known in the art, and any method may be employed in the present invention to prepare such antibodies and equivalents thereof.

As another method to detect the expression level of C12ORF48 gene based on its translation product, the intensity of staining may be observed via immunohistochemical analysis using an antibody against C12ORF48 protein. Namely, the observation of strong staining indicates increased presence of the protein and at the same time high expression level of C12ORF48 gene.

Moreover, in addition to the expression level of C12ORF48 gene, the expression level of other cancer-associated genes, for example, genes known to be differentially expressed in cancer may also be determined to improve the accuracy of the diagnosis.

The expression level of cancer marker gene including C12ORF48 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of C12ORF48 gene in samples from subjects whose disease state are known. Furthermore, the control level can be a database of expression patterns from previously tested cells. Moreover, according to an aspect of the present invention, the expression level of C12ORF48 gene in a biological sample may be compared to multiple control levels, which control levels are determined from multiple reference samples. It is preferred to use a control level determined from a reference sample derived from a tissue type similar to that of the subject-derived biological sample. Moreover, it is preferred, to use the standard value of the expression levels of C12ORF48 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known to be non-cancerous is referred to as a “normal control level”. On the other hand, if the control level is determined from a cancerous biological sample, it is referred to as a “cancerous control level”.

When the expression level of C12ORF48 gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference that is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

A Kit for Diagnosing Cancer

The present invention provides a kit for diagnosing cancer, which may also be useful in assessing the prognosis of cancer and/or monitoring the efficacy of a cancer therapy. Preferably, the cancer is pancreatic cancer or prostate cancer. More particularly, the kit preferably includes at least one reagent for detecting the expression of the C12ORF48 gene in a subject-derived biological sample, which reagent may be selected from the group of:

(a) a reagent for detecting mRNA of the C12ORF48 gene;

(b) a reagent for detecting the C12ORF48 protein; and

(c) a reagent for detecting the biological activity of the C12ORF48 protein.

Suitable reagents for detecting mRNA of the C12ORF48 gene include nucleic acids that specifically bind to or identify the C12ORF48 mRNA, such as oligonucleotides which have a complementary sequence to a part of the C12ORF48 mRNA. These kinds of oligonucleotides are exemplified by primers and probes that are specific to the C12ORF48 mRNA. These kinds of oligonucleotides may be prepared based on methods well known in the art. If needed, the reagent for detecting the C12ORF48 mRNA may be immobilized on a solid matrix. Moreover, more than one reagent for detecting the C12ORF48 mRNA may be included in the kit.

On the other hand, suitable reagents for detecting the C12ORF48 protein include antibodies to the C12ORF48 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the C12ORF48 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the C12ORF48 protein may be included in the kit.

Furthermore, the biological activity can be determined by, for example, measuring the cell proliferating activity due to the expressed C12ORF48 protein in the biological sample. For example, the cell is cultured in the presence of a subject-derived biological sample, and then by detecting the speed of proliferation, or by measuring the cell cycle or the colony forming ability the cell proliferating activity of the biological sample can be determined. If needed, the reagent for detecting the C12ORF48 mRNA may be immobilized on a solid matrix. Moreover, more than one reagent for detecting the biological activity of the C12ORF48 protein may be included in the kit.

The kit may contain more than one of the aforementioned reagents. Furthermore, the kit may include a solid matrix and reagent for binding a probe against the C12ORF48 gene or antibody against the C12ORF48 protein, a medium and container for culturing cells, positive and negative control reagents, and a secondary antibody for detecting an antibody against the C12ORF48 protein. For example, tissue samples obtained from subject suffering from cancer or not may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

As an embodiment of the present invention, when the reagent is a probe against the C12ORF48 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of C12ORF48 mRNA present in the sample. The detection sites may be configured in any suitably detectable shape and are typically in the shape of a bar or dot spanning the width of a test strip.

The kit of the present invention may further include a positive control sample or C12ORF48 standard sample. The positive control sample of the present invention may be prepared by collecting C12ORF48 positive samples and then those C12ORF48 level are assayed. Alternatively, purified C12ORF48 protein or polynucleotide may be added to cells non-expressing C12ORF48 to form the positive sample or the C12ORF48 standard. In the present invention, purified C12ORF48 may be recombinant protein. The C12ORF48 level of the positive control sample is, for example, more than cut off value.

Screening for an Anti-Cancer Compound

In the context of the present invention, agents to be identified through the present screening methods include any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozyme, aptamer, etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents useful in the screenings described herein can also be antibodies that specifically bind to a C12ORF48 protein or partial peptides thereof that lack the biological activity of the original proteins in vivo.

Although the construction of test agent libraries is well known in the art, herein below, additional guidance in identifying test agents and construction libraries of such agents for the present screening methods are provided.

(i) Molecular Modeling

Construction of test agent libraries is facilitated by knowledge of the molecular structure of compounds known to have the properties sought, and/or the molecular structure of C12ORF48. One approach to preliminary screening of test agents suitable for further evaluation utilizes computer modeling of the interaction between the test agent and its target.

Computer modeling technology allows for the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menudriven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles have been published on the subject of computer modeling of drugs interactive with specific proteins, examples of which include Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents suited to the treatment and/or prophylaxis of cancer and/or the prevention of post-operative recurrence of cancer, particularly wherein, such as pancreatic cancer and prostate cancer.

(ii) Combinatorial Chemical Synthesis

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides with six amino acids in length. Such a peptide library could include every 6 amino acid sequence permutation. This type of library is termed a linear combinatorial chemical library.

Preparation of Combinatorial Chemical Libraries is Well Known to Those of Skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31.; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(iii) Other Candidates

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) disclose SELEX (Systematic Evolution of Ligands by Exponential Enrichment) method for selection of aptamers. In the SELEX method, a large library of nucleic acid molecules (e.g., 10¹⁵ different molecules) can be used for screening.

Screening for a C12ORF48 Binding Compound

In context of the present invention, over-expression of C12ORF48 was detected in pancreatic cancer and prostate cancer, in spite of no expression in normal organs (FIG. 1). Accordingly, using the C12ORF48 genes and proteins encoded by the genes, the present invention provides a method of screening for a compound that binds to C12ORF48. Due to the expression of C12ORF48 in cancer, a compound binds to

C12ORF48 is expected to suppress the proliferation of cancer cells, and thus be useful for treating or preventing cancer. Therefore, the present invention also provides a method of screening for a compound that suppresses the proliferation of cancer cells, and a method for screening a compound for treating or preventing cancer using the C12ORF48 polypeptide, wherein the cancer is pancreatic cancer or prostate cancer. One particular embodiment of this screening method includes the steps of:

(a) contacting a test compound with a polypeptide encoded by a polynucleotide of C12ORF48;
(b) detecting the binding activity between the polypeptide and the test compound; and
(c) selecting the test compound that binds to the polypeptide.

In the context of the present invention, the therapeutic effect may be correlated with the binding level of the test agent or compound and C12ORF48 proteins. For example, when the test agent or compound bind to a C12ORF48 protein, the test agent or compound may identified or selected as the candidate agent or compound having the requisite therapeutic effect. Alternatively, when the test agent or compound does not binds to an C12ORF48 protein, the test agent or compound may identified as the agent or compound having no significant therapeutic effect.

The method of the present invention will be described in more detail below.

The C12ORF48 polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the C12ORF48 polypeptide using the C12ORF48 polypeptide, many methods well known by a person skilled in the art can be used. Such a screening can be conducted by, for example, immunoprecipitation method, specifically, in the following manner. The gene encoding the C12ORF48 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EFalpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by the C12ORF48 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green fluorescent protein (GFP) and so on by the use of its multiple cloning sites are commercially available. Also, a fusion protein prepared by introducing only small epitopes consisting of several to a dozen amino acids so as not to change the property of the C12ORF48 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the C12ORF48 polypeptide (Experimental Medicine 13: 85-90 (1995)).

In immunoprecipitation, an immune complex is formed by adding these antibodies to cell lysate prepared using an appropriate detergent. The immune complex consists of the C12ORF48 polypeptide, a polypeptide including the binding ability with the polypeptide, and an antibody. Immunoprecipitation can be also conducted using antibodies against the C12ORF48 polypeptide, besides using antibodies against the above epitopes, which antibodies can be prepared as described above. An immune complex can be precipitated, for example, by Protein A sepharose or Protein G sepharose when the antibody is a mouse IgG antibody. If the polypeptide encoded by C12ORF48 gene is prepared as a fusion protein with an epitope, such as GST, an immune complex can be formed in the same manner as in the use of the antibody against the C12ORF48 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the C12ORF48 polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

West-Western blotting analysis (Skolnik et al., Cell 65: 83-90 (1991)) can be used as a method of screening for proteins binding to the C12ORF48 polypeptide using the polypeptide. In particular, a protein binding to the C12ORF48 polypeptide can be obtained by preparing a cDNA library from cultured cells expected to express a protein binding to the C12ORF48 polypeptide using a phage vector (e.g., ZAP), expressing the protein on LB-agarose, fixing the protein expressed on a filter, reacting the purified and labeled C12ORF48 polypeptide with the above filter, and detecting the plaques expressing proteins bound to the C12ORF48 polypeptide according to the label. The polypeptide of the invention may be labeled by utilizing the binding between biotin and avidin, or by utilizing an antibody that specifically binds to the C12ORF48, or a peptide or polypeptide (for example, GST) that is fused to the C12ORF48 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the polypeptide of the invention is fused to the SRFbinding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by C12ORF48 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized C12ORF48 polypeptide is exposed to synthetic chemical compounds, or natural substance banks or a random phage peptide display library, and the methods of screening using highthroughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the C12ORF48 protein (including agonist and antagonist) are well known to one skilled in the art.

Screening for a Compound that Suppresses the Biological Activity of C12ORF48

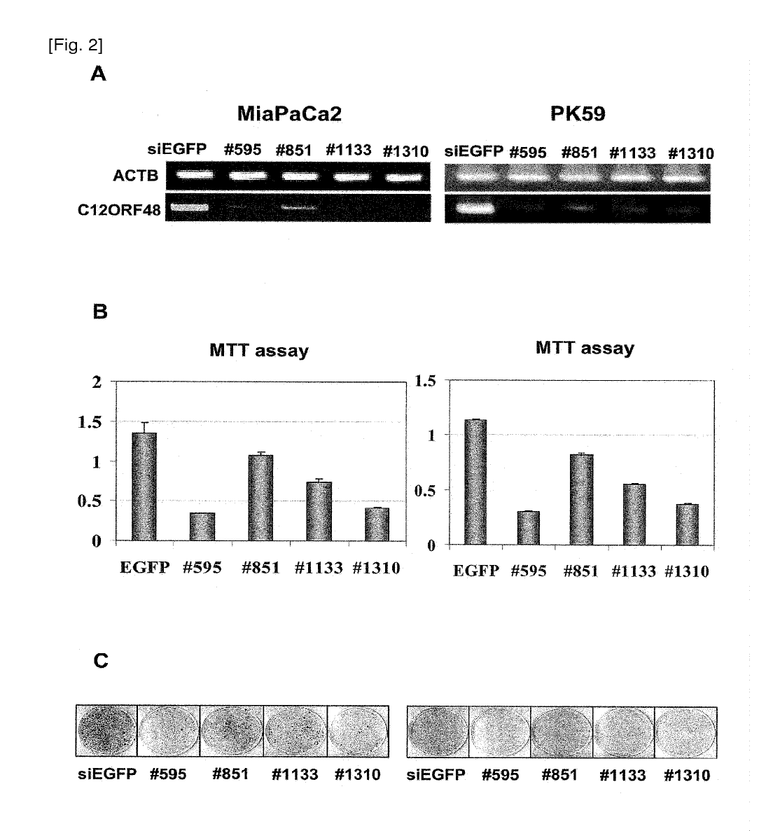

In the context of the present invention, the C12ORF48 protein is characterized as having the activity of promoting cell proliferation of cancer cells (FIG. 2). Using this biological activity as an index, the present invention provides a method for screening a compound that suppresses the proliferation of cancer cells expressing C12ORF48, and a method of screening for a compound for treating or preventing cancer, particularly cancers including pancreatic cancer and prostate cancer. Thus, the present invention provides a method of screening for a compound for treating or preventing cancer using the polypeptide encoded by C12ORF48 gene including the steps as follows:

(a) contacting a test compound with a polypeptide encoded by a polynucleotide of C12ORF48;

(b) detecting the biological activity of the polypeptide of step (a); and

(c) selecting the test compound that suppresses the biological activity of the polypeptide encoded by the polynucleotide of C12ORF48 as compared to the biological activity of the polypeptide detected in the absence of the test compound.

According to the present invention, the therapeutic effect of the test compound on suppressing the activity to promote cell proliferation, or a candidate compound for treating or preventing cancer relating to C12ORF48 (e.g., pancreatic cancer and prostate cancer.) may be evaluated. Therefore, the present invention also provides a method of screening for a candidate compound for suppressing the cell proliferation, or a candidate compound for treating or preventing cancer relating to C12ORF48, using the C12ORF48 polypeptide or fragments thereof including the steps as follows:

a) contacting a test compound with the C12ORF48 polypeptide or a functional fragment thereof;

b) detecting the biological activity of the polypeptide or fragment of step (a), and

c) correlating the biological activity of b) with the therapeutic effect of the test agent or compound.

In the context of the present invention, the therapeutic effect may be correlated with the biological activity of a C12ORF48 polypeptide or a functional fragment thereof. For example, when the test agent or compound suppresses or inhibits the biological activity of a C12ORF48 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity of a C12ORF48 polypeptide or a functional fragment thereof as compared to a level detected in the absence of the test agent or compound, the test agent or compound may identified as the agent or compound having no significant therapeutic effect.

The method of the present invention will be described in more detail below.

Any polypeptides can be used for screening so long as they suppress a biological activity of a C12ORF48 protein. Such biological activity includes cell-proliferating activity the C12ORF48 protein. For example, C12ORF48 protein can be used and polypeptides functionally equivalent to these proteins can also be used. Such polypeptides may be expressed endogenously or exogenously by cells.

The compound isolated by this screening is a candidate for antagonists of the polypeptide encoded by C12ORF48 gene. The term “antagonist” refers to molecules that inhibit the function of the polypeptide by binding thereto. This term also refers to molecules that reduce or inhibit expression of the gene encoding C12ORF48. Moreover, a compound isolated by this screening is a candidate for compounds which inhibit the in vivo interaction of the C12ORF48 polypeptide with molecules (including DNAs and proteins).

When the biological activity to be detected in the present method is cell proliferation, it can be detected, for example, by preparing cells which express the C12ORF48 polypeptide, culturing the cells in the presence of a test compound, and determining the speed of cell proliferation, measuring the cell cycle and such, as well as by measuring survival cells or the colony forming activity, for example, shown in FIG. 2.

The compounds that reduce the speed of proliferation of the cells expressed C12ORF48 are selected as candidate compound for treating or preventing pancreatic cancer and prostate cancer.

More specifically, the method includes the steps of:

(a) contacting a test compound with cells over-expressing C12ORF48;

(b) measuring cell-proliferating activity; and

(c) selecting the test compound that reduces the cell-proliferating activity in the comparison with the cell-proliferating activity in the absence of the test compound.

In preferable embodiments, the method of the present invention may further include the step of:

(d) selecting the test compound that have no effect to the cells no or little expressing C12ORF48.

The phrase “suppress or reduce the biological activity” as defined herein are preferably at least 10% suppression of the biological activity of C12ORF48 in comparison with in absence of the compound, more preferably at least 25%, 50% or 75% suppression and most preferably at 90% suppression.

Screening Using the Binding of C12ORF48 and PARP1 as an Index

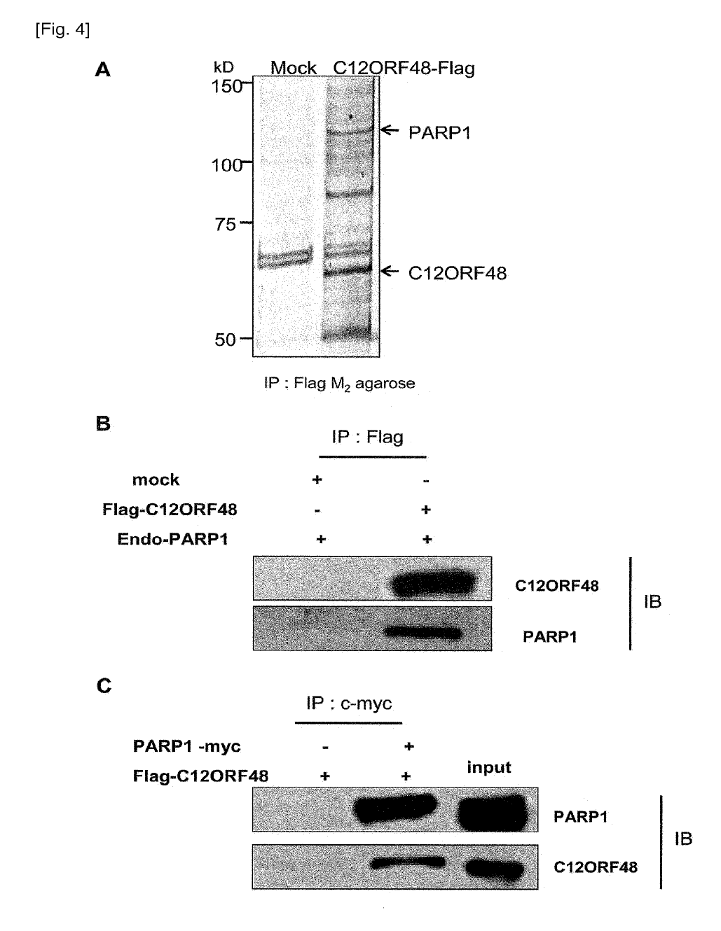

In the present invention, it was confirmed that the C12ORF48 protein interacts with PARP1 protein (FIG. 4). Thus, a compound that inhibits the binding between C12ORF48 protein and PARP1 protein can be screened using such a binding of C12ORF48 protein and PARP1 protein as an index. Therefore, the present invention provides a method for screening a compound for inhibiting the binding between C12ORF48 protein and PARP1 protein can be screened using such a binding of C12ORF48 protein and PARP1 protein as an index. Furthermore, the present invention also provides a method for screening a compound for inhibiting or reducing a growth of cancer cells expressing C12ORF48, e.g. pancreatic cancer cell and prostate cancer cell, and a compound for treating or preventing cancers, e.g. pancreatic cancer or prostate cancer.

Specifically, the present invention provides the following methods of [1] to [5]:

[1] A method of screening for an agent or compound that interrupts a binding between a C12ORF48 polypeptide and a PARP1 polypeptide, the method comprising the steps of:

(a) contacting a C12ORF48 polypeptide or functional equivalent thereof with a PARP1 polypeptide or functional equivalent thereof in the presence of a test agent or compound;

(b) detecting a binding between the polypeptides;

(c) comparing the binding level detected in the step (b) with those detected in the absence of the test agent or compound; and

(d) selecting the test agent or compound that reduce or inhibits the binding level.

[2] A method of screening for an agent or compound useful in treating or preventing cancers, the method comprising the steps of:

(a) contacting a C12ORF48 polypeptide or functional equivalent thereof with a PARP1 polypeptide or functional equivalent thereof in the presence of a test agent or compound;

(b) detecting a binding between the polypeptides;

(c) comparing the binding level detected in the step (b) with those detected in the absence of the test agent or compound; and

(d) selecting the test agent or compound that reduce or inhibits the binding level.

[3] The method of [1] or [2], wherein the functional equivalent of C12ORF48 comprising the PARP1-binding domain.

[4] The method of [1] or [2], wherein the functional equivalent of PARP1 comprising the C12ORF48-binding domain.

[5] The method of [1], wherein the cancer is selected from the group consisting of pancreatic cancer and prostate cancer.

According to the present invention, the therapeutic effect of the test agent or compound on inhibiting the cell growth or a candidate agent or compound for treating or preventing C12ORF48 associating disease may be evaluated. Therefore, the present invention also provides a method for screening a candidate agent or compound that suppresses the proliferation of cancer cells, and a method for screening a candidate agent or compound for treating or preventing cancer.

More specifically, the method includes the steps of:

(a) contacting a C12ORF48 polypeptide, or functional equivalent thereof, with a PARP1 polypeptide, or functional equivalent thereof, in the presence of a test agent or compound;

(b) detecting the level of binding between the polypeptides; and

(c) comparing the binding level of the C12ORF48 and PARP1 proteins with that detected in the absence of the test agent or compound; and

(d) correlating the binding level of c) with the therapeutic effect of the test agent or compound.

In the context of the present invention, a functional equivalent of an C12ORF48 or PARP1 polypeptide is a polypeptide that has a biological activity equivalent to a C12ORF48 polypeptide (SEQ ID NO: 11) or PARP1 (SEQ ID NO: 13) polypeptide, respectively.

As a method of screening for compounds that modulates, e.g. inhibits, the binding of C12ORF48 to PARP1, many methods well known by one skilled in the art can be used.

A polypeptide to be used for screening can be a recombinant polypeptide or a protein derived from natural sources, or a partial peptide thereof. Any test compound aforementioned can be used for screening.

As a method of screening for proteins, for example, that bind to a polypeptide using C12ORF48 or PARP1 polypeptide or functionally equivalent thereof, many methods well known by a person skilled in the art can be used. Such a screening can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”), affinity chromatography and A biosensor using the surface plasmon resonance phenomenon. Any aforementioned test compound can be used. In some embodiments, this method further comprises the step of detecting the binding of the candidate compound to C12ORF48 protein or PARP1 protein, or detecting the level of binding C12ORF48 protein to or PARP1 protein. Cells expressing C12ORF48 protein and/or PARP1 proteins include, for example, cell lines established from cancer, e.g. lung cancer such cells can be used for the above screening of the present invention so long as the cells express these two genes. Alternatively cells can be transfected both or either of expression vectors of C12ORF48 and PARP1 protein, so as to express these two genes. The binding of C12ORF48 protein to PARP1 protein can be detected by immunoprecipitation assay using an anti-C12ORF48 antibody and PARP1 antibody (FIG. 4).

Screening for a Compound that Suppresses the Biological Activity of PARP1

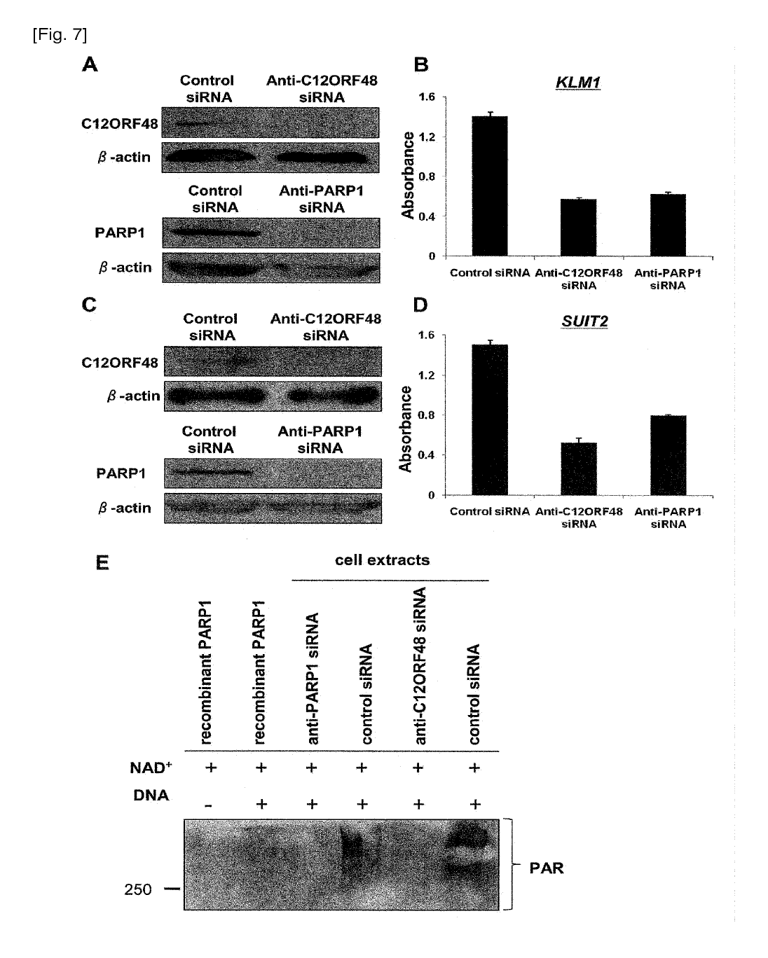

In the context of the present invention, it was confirmed that PARP1 activity was drastically decreased in C12ORF48 knockdown (FIG. 7E). Using this activity as an index, the present invention provides a method for screening a compound that suppresses the proliferation of cancer cells expressing C12ORF48 and PARP1, and a method of screening for a compound for treating or preventing cancer, particularly cancers including pancreatic cancer and prostate cancer. Thus, the present invention provides a method of screening for a compound for treating or preventing cancer using the polypeptide encoded by C12ORF48 gene including the steps as follows:

(a) contacting a test compound with a polypeptide encoded by a polynucleotide of C12ORF48 in the presence of a polypeptide encoded by a polynucleotide PARP1;
(b) detecting the biological activity of the polypeptide encoded by a polynucleotide of PARP1; and
(c) selecting the test compound that suppresses the biological activity of the polypeptide encoded by the polynucleotide of PARP1 as compared to the biological activity of the polypeptide detected in the absence of the test compound.

According to the present invention, the therapeutic effect of the test compound on suppressing the automodification activity of PARP1, or a candidate compound for treating or preventing cancer relating to C12ORF48 (e.g., pancreatic cancer and prostate cancer.) may be evaluated. Therefore, the present invention also provides a method of screening for a candidate compound for suppressing the automodification activity, or a candidate compound for treating or preventing cancer relating to C12ORF48, using the C12ORF48 polypeptide or fragments thereof and PARP1 polypeptide or fragments thereof including the steps as follows:

a) contacting a test compound with the C12ORF48 polypeptide or a functional fragment thereof in the presence of the PAPP1 polypeptide or a functional fragment thereof;

b) detecting the biological activity of the PAPP1 polypeptide or fragment of step (a), and

c) correlating the biological activity of b) with the therapeutic effect of the test agent or compound.

In the context of the present invention, the therapeutic effect may be correlated with the automodulation activity of a PARP1 polypeptide or a functional fragment thereof enhanced by C12ORF48. For example, when the test agent or compound suppresses or inhibits the automodulation activity of PARP1 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the automodulation activity of a PARP1 polypeptide or a functional fragment thereof as compared to a level detected in the absence of the test agent or compound, the test agent or compound may identified as the agent or compound having no significant therapeutic effect.

The method of the present invention will be described in more detail below.

Any polypeptides can be used for screening so long as they suppress an automodulation activity of a PARP1 protein. For example, C12ORF48 protein and PARP1 protein can be used and polypeptides functionally equivalent to these proteins can also be used. Such polypeptides may be expressed endogenously or exogenously by cells.

The compound isolated by this screening is a candidate for antagonists of the polypeptide encoded by C12ORF48 gene. The term “antagonist” refers to molecules that inhibit the function of the polypeptide by binding thereto. This term also refers to molecules that reduce or inhibit expression of the gene encoding C12ORF48. Moreover, a compound isolated by this screening is a candidate for compounds which inhibit the in vivo interaction of the C12ORF48 polypeptide with PARP1.

When the biological activity to be detected in the present method is automodulation, it can be detected, for example, by preparing cells which express the C12ORF48 and PARP1 polypeptide, culturing the cells in the presence of a test compound, and determining the automodulation of PARP1, measuring the cell cycle and such, as well as by measuring survival cells or the colony forming activity. The compounds that reduce the automodulation of PARP1 of the cells expressed C12ORF48 are selected as candidate compound for treating or preventing pancreatic cancer and prostate cancer.

More specifically, the method includes the steps of:

(a) contacting a test compound with cells over-expressing C12ORF48 and PARP1;

(b) measuring the automodulation activity of PARP1; and

(c) selecting the test compound that reduces the automodulation activity in the comparison with the cell-proliferating activity in the absence of the test compound.

In preferable embodiments, the method of the present invention may further include the step of:

(d) selecting the test compound that have no effect to the cells no or little expressing C12ORF48.

The phrase “suppress or reduce the automodulation” as defined herein are preferably at least 10% suppression of the biological activity of C12ORF48 in comparison with in absence of the compound, more preferably at least 25%, 50% or 75% suppression and most preferably at 90% suppression.

Screening for a Compound Altering the Expression of C12ORF48

In the present invention, a decrease in the expression of C12ORF48 by siRNA results in the inhibition of cancer cell proliferation (FIG. 2). Accordingly, the present invention provides a method of screening for a compound that inhibits the expression of C12ORF48. A compound that inhibits the expression of C12ORF48 is expected to suppress the proliferation of cancer cells, and thus is useful for treating or preventing cancer, particularly cancers such as pancreatic cancer and prostate cancer. Therefore, the present invention also provides a method for screening a compound that suppresses the proliferation of cancer cells, and a method for screening a compound for treating or preventing cancer. In the context of the present invention, such screening may include, for example, the following steps:

(a) contacting a candidate compound with a cell expressing C12ORF48; and
(b) selecting the candidate compound that reduces the expression level of C12ORF48 as compared to a control.

According to the present invention, the therapeutic effect of the test agent or compound on inhibiting the cell growth or a candidate agent or compound for treating or preventing C12ORF48 associating disease may be evaluated. Therefore, the present invention also provides a method for screening a candidate agent or compound that suppresses the proliferation of cancer cells, and a method for screening a candidate agent or compound for treating or preventing C12ORF48 associating disease.

In the context of the present invention, such screening may include, for example, the following steps:

a) contacting a test agent or compound with a cell expressing the C12ORF48 gene;

b) detecting the expression level of the C12ORF48 gene; and

c) correlating the expression level of b) with the therapeutic effect of the test agent or compound.

In the context of present invention, the therapeutic effect may be correlated with the expression level of the C12ORF48 gene. For example, when the test agent or compound reduces the expression level of the C12ORF48 gene as compared to a level detected in the absence of the test agent or compound, the test agent or compound may identified or selected as the candidate agent or compound having the therapeutic effect. Alternatively, when the test agent or compound does not reduce the expression level of the C12ORF48 gene as compared to a level detected in the absence of the test agent or compound, the test agent or compound may identified as the agent or compound having no significant therapeutic effect.

The method of the present invention will be described in more detail below.

Cells expressing the C12ORF48 include, for example, cell lines established from pancreatic cancer and prostate cancer or cell lines transfected with C12ORF48 expression vectors; any of such cells can be used for the above screening of the present invention. The expression level can be estimated by methods well known to one skilled in the art, for example, RT-PCR, Northern blot assay, Western blot assay, immunostaining and flow cytometry analysis. “Reduce the expression level” as defined herein are preferably at least 10% reduction of expression level of C12ORF48 in comparison to the expression level in absence of the compound, more preferably at least 25%, 50% or 75% reduced level and most preferably at 95% reduced level. The compound herein includes chemical compound, double-strand nucleotide, and so on. The preparation of the double-strand nucleotide is in aforementioned description. In the method of screening, a compound that reduces the expression level of C12ORF48 can be selected as candidate compounds to be used for the treatment or prevention of pancreatic cancer and prostate cancer.

Alternatively, the screening method of the present invention may include the following steps:

(a) contacting a candidate compound with a cell into which a vector, including the transcriptional regulatory region of C12ORF48 and a reporter gene that is expressed under the control of the transcriptional regulatory region, has been introduced;

(b) measuring the expression or activity of the reporter gene; and

(c) selecting the candidate compound that reduces the expression or activity of the reporter gene.

According to the present invention, the therapeutic effect of the test agent or compound on inhibiting the cell growth or a candidate agent or compound for treating or preventing C12ORF48 associating disease may be evaluated. Therefore, the present invention also provides a method for screening a candidate agent or compound that suppresses the proliferation of cancer cells, and a method for screening a candidate agent or compound for treating or preventing a C12ORF48 associated disease.

In the context of the present invention, such screening may include, for example, the following steps:

a) contacting a test agent or compound with a cell into which a vector, composed of the transcriptional regulatory region of the C12ORF48 gene and a reporter gene that is expressed under the control of the transcriptional regulatory region, has been introduced;

b) detecting the expression or activity of the reporter gene; and

c) correlating the expression level of b) with the therapeutic effect of the test agent or compound.

In the context of the present invention, the therapeutic effect may be correlated with the expression or activity of the reporter gene. For example, when the test agent or compound reduces the expression or activity of the reporter gene as compared to a level detected in the absence of the test agent or compound, the test agent or compound may identified or selected as the candidate agent or compound having the therapeutic effect. Alternatively, when the test agent or compound does not reduce the expression or activity of the reporter gene as compared to a level detected in the absence of the test agent or compound, the test agent or compound may identified as the agent or compound having no significant therapeutic effect.

Suitable reporter genes and host cells are well known in the art. Illustrative reporter genes include, but are not limited to, luciferase, green fluorescent protein (GFP), Discosoma sp. Red Fluorescent Protein (DsRed), Chrolamphenicol Acetyltransferase (CAT), lacZ and beta-glucuronidase (GUS), and host cell is COS7, HEK293, HeLa and so on. The reporter construct required for the screening can be prepared by connecting reporter gene sequence to the transcriptional regulatory region of C12ORF48. The transcriptional regulatory region of C12ORF48 herein is the region from transcriptional start site to at least 500 bp upstream, preferably 1,000 bp, more preferably 5,000 or 10,000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the reporter construct is infected to host cells and the expression or activity of the reporter gene is detected by method well known in the art (e.g., using luminometer, absorption spectrometer, flow cytometer and so on). “Reduces the expression or activity” as defined herein are preferably at least 10% reduction of the expression or activity of the reporter gene in comparison with in absence of the compound, more preferably at least 25%, 50% or 75% reduction and most preferably at 95% reduction.

In the context of the present invention, candidate compounds that have the potential to treat or prevent cancers can be identified. The therapeutic potential of these candidate compounds may be evaluated by second and/or further screening to identify therapeutic agent for cancers. For example, when a compound binding to C12ORF48 protein inhibits activities of the cancer described above, it may be concluded that such compound has the C12ORF48 specific therapeutic effect.

Double Stranded Molecule

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a C12ORF48 sense nucleic acid sequence (also referred to as “sense strand”), a C12ORF48 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of the first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As used herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a C12ORF48 sense nucleic acid sequence (also referred to as “sense strand”), a C12ORF48 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the context of the present invention, examples of isolated nucleic acid include DNA, RNA, and derivatives thereof.

A double-stranded molecule against C12ORF48 that hybridizes to target mRNA, decreases or inhibits production of C12ORF48 protein encoded by C12ORF48 gene by associating with the normally single-stranded mRNA transcript of the gene, thereby interfering with translation and thus, inhibiting expression of the protein. As demonstrated herein, the expression of C12ORF48 in pancreatic cancer cell lines was inhibited by dsRNA (FIG. 2). Accordingly, the present invention provides isolated double-stranded molecules that are capable of inhibiting the inhibit expression of a C12ORF48 gene when introduced into a cell expressing the gene. The target sequence of double-stranded molecule may be designed by an siRNA design algorithm such as that mentioned below.

Examples of C12ORF48 target sequences include, for example, nucleotides such as

SEQ ID NO: 5 (at the position 595-613 nt of SEQ ID NO: 10)

SEQ ID NO: 7 (at the position 1133-1151 nt of SEQ ID NO: 10)

SEQ ID NO: 8 (at the position 1310-1328 nt of SEQ ID NO: 10)

SEQ ID NO: 14 (at the position 606-624 of SEQ ID NO: 10)

In the same way a double-stranded molecule against PARP1 that hybridizes to target mRNA, decreases or inhibits production of PARP1 protein encoded by PARP1 gene by associating with the normally single-stranded mRNA transcript of the gene, thereby interfering with translation and thus, inhibiting expression of the protein. As demonstrated herein, the expression of PARP1 in pancreatic cancer cell lines was inhibited by dsRNA (FIG. 7). Accordingly, the present invention provides isolated double-stranded molecules that are capable of inhibiting the inhibit expression of a PARP1 gene when introduced into a cell expressing the gene. The target sequence of double-stranded molecule may be designed by an siRNA design algorithm such as that mentioned below.

Examples of PARP1 target sequences include, for example, nucleotides such as SEQ ID NO: 15 (at the position 2685-2703 nt of SEQ ID NO: 12)

Of particular interest in the present invention are the following double-stranded molecules [1] to [18]:

[1] An isolated double-stranded molecule that, when introduced into a cell, inhibits in vivo expression of C12ORF48 or PARPland cell proliferation, such molecules composed of a sense strand and an antisense strand complementary thereto, hybridized to each other to form the double-stranded molecule;

[2] The double-stranded molecule of [1], wherein the double-stranded molecule acts on mRNA, matching a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10), SEQ ID NO: 14 (at the position 606-624 of SEQ ID NO: 10) and SEQ ID NO: 15 (at the position 2685-2703 nt of SEQ ID NO: 12);

[3] The double-stranded molecule of [2], wherein the sense strand contains a sequence corresponding to a target sequence selected from among SEQ ID NOs: 5, 7, 8, 14 and 15;

[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6], having a length of less than about 25 nucleotides;

[8] The double-stranded molecule of [7], having a length of between about 19 and about 25 nucleotides;

[9] The double-stranded molecule of [1], composed of a single polynucleotide having both the sense and antisense strands linked by an intervening single-strand;

[10] The double-stranded molecule of [9], having the general formula 5′-[A]-[B]-[A′]-3′, wherein [A] is the sense strand containing a sequence corresponding to a target sequence selected from among SEQ ID NOs: 5, 7, 8, 14 and 15, [B] is the intervening single-strand composed of 3 to 23 nucleotides, and [A′] is the antisense strand containing a sequence complementary to [A];

[11] The double-stranded molecule of [1], composed of RNA;

[12] The double-stranded molecule of [1], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [2], wherein the molecule contains 3′ overhang. The double-stranded molecule of the present invention will be described in more detail below.

Methods for designing double-stranded molecules having the ability to inhibit target gene expression in cells are known (See, for example, U.S. Pat. No. 6,506,559, herein incorporated by reference in its entirety). For example, a computer program for designing siRNAs is available from the Ambion website (www.ambion.com/techlib/misc/siRNA finder.html).

The computer program selects target nucleotide sequences for double-stranded molecules based on the following protocol.

Selection of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTRbinding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention was designed as: SEQ ID NOs: 5, 7, 8 and 14 for C12ORF48 gene and SEQ ID NOs: 15 for PARP1

Double-stranded molecules targeting the above-mentioned target sequences were respectively examined for their ability to suppress the growth of cells expressing the target genes. Therefore, the present invention provides double-stranded molecules targeting any of the sequences selected from the group of:

SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10) and SEQ ID NO: 14 (at the position 606-624 of SEQ ID NO: 10) for C12ORF48 gene, and SEQ ID NO: 15 (at the position 2685-2703 nt of SEQ ID NO: 12) for PARP1.

The double-stranded molecule of the present invention may be directed to a single target C12ORF48 or PARP1 gene sequence or may be directed to a plurality of target C12ORF48 or PARP1 gene sequences.

A double-stranded molecule of the present invention targeting the above-mentioned targeting sequence of C12ORF48 or PARP1 gene include isolated polynucleotides that contain any of the nucleic acid sequences of target sequences and/or complementary sequences to the target sequences. Examples of polynucleotides targeting C12ORF48 or PARP1 gene include those containing the sequence of SEQ ID NOs: 5, 7, 8, 14 and 15 and/or complementary sequences to these nucleotides. However, the present invention is not limited to these examples, and minor modifications in the aforementioned nucleic acid sequences are acceptable so long as the modified molecule retains the ability to suppress the expression of C12ORF48 or PARP1 gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of C12ORF48 or PARP1 genes or antisense strands complementary thereto were tested in vitro for their ability to decrease production of a C12ORF48 or PARP1 gene product in pancreatic cancer cell lines (e.g., using MiaPaCa2, PK59, KLM1 and SUIT2) according to standard methods. Furthermore, for example, reduction in a C12ORF48 or PARP1 gene product in cells contacted with the candidate double-stranded molecule compared to cells cultured in the absence of the candidate molecule can be detected by, e.g. RT-PCR using primers for the C12ORF48 or PARP1 mRNA mentioned under Example item “Semi-quantitative RT-PCR”. Sequences that decrease the production of a C12ORF48 or PARP1 gene product in vitro cell-based assays can then be tested for their inhibitory effects on cell growth. Sequences that inhibit cell growth in vitro cell-based assay can then be tested for their in vivo ability using animals with cancer, e.g. nude mouse xenograft models, to confirm decreased production of a C12ORF48 or PARP1 gene product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 3,189 nucleotides in length for C12ORF48 and less than 4,001 nucleotides in length for PARP1. For example, the polynucleotide is less than 500, 200, 100, 75, 50, or 25 nucleotides in length for all of the genes. The isolated polynucleotides of the present invention are useful for forming double-stranded molecules against C12ORF48 or PARP1 gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides. Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C— methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or vice versa, so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:
5′-[---DNA---]-3′
3′-(RNA)-[DNA]-5′
:antisense strand,
sense strand:
5′-(RNA)-[DNA]-3′
3′-(RNA)-[DNA]-5′
:antisense strand,
and
sense strand:
5′-(RNA)-[DNA]-3′
3′-(---RNA---)-5′
:antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the context of the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotides of SEQ ID NOs: 5, 7, 8 and 14 for C12ORF48, and of SEQ ID NO: 15 for PARP1.

The present invention is not limited to these examples, and the target sequence in [A] may be modified sequences from these examples so long as the double-stranded molecule retains the ability to suppress the expression of the targeted C12ORF48 or PARP1 gene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

5′-CACAGUAUCUCCUAGUCAA-[B]-UUGACUAGGAGAUACUGUG-3′ (for target sequence SEQ ID NO: 5);
5′-GUUGCUCAGGAUUUGGAUU-[B]-AAUCCAAAUCCUGAGCAAC-3′ (for target sequence of SEQ ID NO: 7);
5′-CUAGUCAACUACUGGAUUU-[B]-AAAUCCAGUAGUUGACUAG-3′ (for target sequence of SEQ ID NO: 14); and
5′-GAUAGAGCGUGAAGGCGAA-[B]-UUCGCCUUCACGCUCUAUC-3′ (for target sequence of SEQ ID NO: 15).

Furthermore, in order to enhance the inhibition activity of the double-stranded molecules, nucleotide “u” can be added to 3′ end of the antisense strand of the target sequence, as 3′ overhangs. The number of “u”s to be added is at least 2, generally 2 to 10, preferably 2 to 5. The added “u”s form single strand at the 3′ end of the antisense strand of the double-stranded molecule.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking C12ORF48 or PARP1 sequences may be identical or different, such that their expression can be modulated independently, or in a temporal or spatial manner. The double-stranded molecules can be transcribed intracellularly by cloning C12ORF48 or PARP1 gene templates into a vector containing, e.g., a RNA pol III transcription unit from the small nuclear RNA (snRNA) U6 or the human H1 RNA promoter.

Vectors containing a double-stranded molecule of the present invention

Also included in the present invention are vectors containing one or more of the double-stranded molecules described herein, and a cell containing such a vector. Of particular interest to the present invention are the following vectors of [1] to [10]:

[1] A vector, encoding a double-stranded molecule that, when introduced into a cell, inhibits in vivo expression of C12ORF48 or PARP1 and cell proliferation, such molecules composed of a sense strand and an antisense strand complementary thereto, hybridized to each other to form the double-stranded molecule.
[2] The vector of [1], encoding the double-stranded molecule acts on mRNA, matching a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10), SEQ ID NO: 14 (at the position 606-624 of SEQ ID NO: 10) and SEQ ID NO: 15 (at the position 2685-2703 nt of SEQ ID NO: 12);
[3] The vector of [1], wherein the sense strand contains a sequence corresponding to a target sequence selected from among SEQ ID NOs: 5, 7, 8, 14 and 15;
[4] The vector of [3], encoding the double-stranded molecule, having a length of less than about 100 nucleotides;
[5] The vector of [4], encoding the double-stranded molecule, having a length of less than about 75 nucleotides;
[6] The vector of [5], encoding the double-stranded molecule, having a length of less than about 50 nucleotides;
[7] The vector of [6] encoding the double-stranded molecule, having a length of less than about 25 nucleotides;
[8] The vector of [7], encoding the double-stranded molecule, having a length of between about 19 and about 25 nucleotides;
[9] The vector of [1], wherein the double-stranded molecule is composed of a single polynucleotide having both the sense and antisense strands linked by an intervening single-strand; and
[10] The vector of [9], encoding the double-stranded molecule having the general formula 5′-[A]-[B]-[A′]-3′, wherein [A] is the sense strand containing a sequence corresponding to a target sequence selected from among SEQ ID NOs: 5, 7 and 8, [B] is the intervening single-strand composed of 3 to 23 nucleotides, and [A′] is the antisense strand containing a sequence complementary to [A].

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning C12ORF48 sequence into an expression vector so that regulatory sequences are operatively-linked to C12ORF48 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vector constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adenoassociated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention

In the present invention, four different dsRNA for C12ORF48 and a dsRNA for PARP1 were tested for their ability to inhibit cell growth. The four dsRNA for C12ORF48 (FIGS. 2 and 7) and the dsRNA for PARP1 (FIG. 7), that effectively knocked down the expression of the gene in pancreatic cancer cell lines coincided with suppression of cell proliferation.

Accordingly, the present invention provides methods for inhibiting cell growth, i.e., pancreatic cancer and prostate cancer cell growth, by inducing dysfunction of the C12ORF48 or PARP1gene via inhibiting the expression of C12ORF48 or PARP1, respectively. C12ORF48 or PARP1 gene expression can be inhibited by any of the aforementioned double-stranded molecules of the present invention that specifically target the C12ORF48 or PARP1gene or the vectors of the present invention that can express any of the double-stranded molecules.

Such ability of the present double-stranded molecules and vectors to inhibit cell growth of cancerous cell indicates that they can be used for methods for treating cancer. Thus, the present invention provides methods to treat patients with pancreatic cancer and prostate cancer by administering a double-stranded molecule against C12ORF48 or PARP1 gene or a vector expressing the molecule without adverse effect because that genes were hardly detected in normal organs (FIG. 1).

Of particular interest to the present invention are the following methods [1] to [36]:

[1] A method for inhibiting growth of cancer cell and treating a cancer, wherein the cancer cell or the cancer expresses at least one gene C12ORF48 gene, such method including the step of administering at least one isolated double-stranded molecule inhibiting the expression of C12ORF48 in a cell over-expressing the gene and the cell proliferation, wherein the double-stranded molecule is composed of a sense strand and an antisense strand complementary thereto, hybridized to each other to form the double-stranded molecule.

[2] The method of [1], wherein the double-stranded molecule acts at mRNA which matches a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10) and SEQ ID NO: 14 (at the position 606-624 of SEQ ID NO: 10) for C12ORF48 gene, and SEQ ID NO: 15 (at the position 2685-2703 nt of SEQ ID NO: 12) for PARP1.

[3] The method of [2], wherein the sense strand contains the sequence corresponding to a target sequence selected from among SEQ ID NOs: 5, 7, 8, 14 and 15.

[4] The method of [1], wherein the cancer to be treated is pancreatic cancer and/or prostate cancer;

[5] The method of [4], wherein the pancreatic cancer is pancreatic ductal adenocarcinoma (PDAC), and the prostate cancer is castration-resistant prostate cancer (CRPC);

[6] The method of [1], wherein plural kinds of the double-stranded molecules are administered;

[7] The method of [3], wherein the double-stranded molecule has a length of less than about 100 nucleotides;

[8] The method of [7], wherein the double-stranded molecule has a length of less than about 75 nucleotides;

[9] The method of [8], wherein the double-stranded molecule has a length of less than about 50 nucleotides;

[10] The method of [9], wherein the double-stranded molecule has a length of less than about 25 nucleotides;

[11] The method of [10], wherein the double-stranded molecule has a length of between about 19 and about 25 nucleotides in length;

[12] The method of [1], wherein the double-stranded molecule is composed of a single polynucleotide containing both the sense strand and the antisense strand linked by an intervening single-strand;

[13] The method of [12], wherein the double-stranded molecule has the general formula 5′-[A]-[B]-[A′]-3′, wherein [A] is the sense strand containing a sequence corresponding to a target sequence selected from among SEQ ID NOs: 5, 7, 8, 14 and 15, [B] is the intervening single strand composed of 3 to 23 nucleotides, and [A′] is the antisense strand containing a sequence complementary to [A];

[14] The method of [1], wherein the double-stranded molecule is an RNA;

[15] The method of [1], wherein the double-stranded molecule contains both DNA and RNA;

[16] The method of [15], wherein the double-stranded molecule is a hybrid of a DNA polynucleotide and an RNA polynucleotide;

[17] The method of [16] wherein the sense and antisense strand polynucleotides are composed of DNA and RNA, respectively;

[18] The method of [15], wherein the double-stranded molecule is a chimera of DNA and RNA;

[19] The method of [18], wherein a region flanking to the 3′-end of the antisense strand, or both of a region flanking to the 5′-end of sense strand and a region flanking to the 3′-end of antisense strand are composed of RNA;

[20] The method of [19], wherein the flanking region is composed of 9 to 13 nucleotides;

[21] The method of [1], wherein the double-stranded molecule contains 3′ overhangs;

[22] The method of [1], wherein the double-stranded molecule is contained in a composition which includes, in addition to the molecule, a transfection-enhancing agent and pharmaceutically acceptable carrier.

[23] The method of [1], wherein the double-stranded molecule is encoded by a vector;

[24] The method of [23], wherein the double-stranded molecule encoded by the vector acts at mRNA which matches a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10) and SEQ ID NO: 14 (at the position 606-624 of SEQ ID NO: 10) for C12ORF48 gene, and SEQ ID NO: 15 (at the position 2685-2703 nt of SEQ ID NO: 12) for PARP1.

[25] The method of [24], wherein the sense strand of the double-stranded molecule encoded by the vector contains the sequence corresponding to a target sequence selected from among SEQ ID NOs: 5, 7, 8, 14 and 15.

[26] The method of [23], wherein the cancer to be treated is pancreatic cancer and/or prostate cancer;

[27] The method of [26], wherein the pancreatic cancer is pancreatic ductal adenocarcinoma (PDAC), and the prostate cancer is castration-resistant prostate cancer (CRPC);

[28] The method of [23], wherein plural kinds of the double-stranded molecules are administered;

[29] The method of [25], wherein the double-stranded molecule encoded by the vector has a length of less than about 100 nucleotides;

[30] The method of [29], wherein the double-stranded molecule encoded by the vector has a length of less than about 75 nucleotides;

[31] The method of [30], wherein the double-stranded molecule encoded by the vector has a length of less than about 50 nucleotides;

[32] The method of [31], wherein the double-stranded molecule encoded by the vector has a length of less than about 25 nucleotides;

[33] The method of [32], wherein the double-stranded molecule encoded by the vector has a length of between about 19 and about 25 nucleotides in length;

[34] The method of [23], wherein the double-stranded molecule encoded by the vector is composed of a single polynucleotide containing both the sense strand and the antisense strand linked by an intervening single-strand;

[35] The method of [34], wherein the double-stranded molecule encoded by the vector has the general formula 5′-[A]-[B]-[A′]-3′, wherein [A] is the sense strand containing a sequence corresponding to a target sequence selected from among SEQ ID NOs: 5, 7, 8, 14 and 15, [B] is a intervening single-strand is composed of 3 to 23 nucleotides, and [A′] is the antisense strand containing a sequence complementary to [A]; and

[36] The method of [23], wherein the double-stranded molecule encoded by the vector is contained in a composition which includes, in addition to the molecule, a transfection-enhancing agent and pharmaceutically acceptable carrier.

The method of the present invention will be described in more detail below.

The growth of cells expressing a C12ORF48 gene may be inhibited by contacting the cells with a double-stranded molecule against a C12ORF48 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell expresses or over-expresses the target gene of the double-stranded molecule of the present invention. Exemplary cells include pancreatic cancer or prostate cancer cells, particularly pancreatic ductal adenocarcinoma (PDAC) or castration-resistant prostate cancer (CRPC).

Thus, patients suffering from or at risk of developing disease related to C12ORF48 may be treated with the administration of at least one of the present double-stranded molecules, at least one vector expressing at least one of the molecules or at least one composition containing at least one of the molecules. For example, patients suffering from pancreatic cancer or prostate cancer may be treated according to the present methods. The type of cancer may be identified by standard methods according to the particular type of tumor to be diagnosed. Preferably, patients treated by the methods of the present invention are selected by detecting the expression of C12ORF48 in a biopsy from the patient by RT-PCR or immunoassay. Preferably, before the treatment of the present invention, the biopsy specimen from the subject is confirmed for C12ORF48 gene over-expression by methods known in the art, for example, immunohistochemical analysis or RT-PCR.

According to the present method to inhibit cell growth and thereby treat cancer, through the administration of plural kinds of the double-stranded molecules (or vectors expressing or compositions containing the same), each of the molecules may have different structures but act on mRNA that matches the same target sequence of C12ORF48 or PARP1. Alternatively plural kinds of the double-stranded molecules may act on mRNA that matches a different target sequence of same gene. Alternatively, for example, the method may utilize double-stranded molecules directed to one, two or more target sequence of C12ORF48 or PARP1.

For inhibiting cell growth, a double-stranded molecule of the present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is deemed “efficacious” if it leads to clinical benefit such as, reduction in expression of C12ORF48 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods and compositions of the present invention find utility in the context of “prevention” and “prophylaxis”, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g., reducing the proliferation and metastasis of tumors.

The treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis includes 10%, 20%, 30% or more reduction, or stable disease.

It is understood that a double-stranded molecule of the present invention degrades C12ORF48 or PARP1 mRNA in substoichiometric amounts. Without wishing to be bound by any theory, it is believed that the double-stranded molecule of the invention causes degradation of the target mRNA in a catalytic manner. Thus, as compared to standard cancer therapies, the present invention requires the delivery of significantly less double-stranded molecule needs at or near the site of cancer in order to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

For treating cancer, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present double-stranded molecule include the Minis Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as pancreatic or prostate tumor tissue, and can also increase the blood half-life of the double-stranded molecule. Liposomes suitable for use in the context of the present invention may be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule includes a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH3 and a solvent mixture such as tetrahydrofuran and water in a 30:12 ratio at 60 degrees C.

Vectors expressing a double-stranded molecule of the present invention are discussed above. Such vectors expressing at least one double-stranded molecule of the invention can also be administered directly or in conjunction with a suitable delivery reagent, including the Mirus Transit LT 1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravesical and intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example, by a catheter or other placement device (e.g., a suppository or an implant including a porous, nonporous, or gelatinous material); and inhalation. It is preferred that injections or infusions of the double-stranded molecule or vector be given at or near the site of the cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

Compositions Containing a Double-Stranded Molecule of the Present Invention

In addition to the above, the present invention also provides pharmaceutical compositions that include at least one of the present double-stranded molecules or the vectors coding for the molecules. Of particular interest to the present invention are the following compositions [1] to [36]:

[1] A composition for inhibiting growth of a cancer cell and treating a cancer, wherein the cancer and the cancer cell express at least one C12ORF48 or PARP1 gene, including at least one isolated double-stranded molecule that inhibits the expression of C12ORF48 or PARP1 and the cell proliferation, further wherein the molecule is composed of a sense strand and an antisense strand complementary thereto, hybridized to each other to form the double-stranded molecule.

[2] The composition of [1], wherein the double-stranded molecule acts at mRNA which matches a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10) and SEQ ID NO: 14 (at the position 606-624 of SEQ ID NO: 10) for C12ORF48 gene, and SEQ ID NO: 15 (at the position 2685-2703 nt of SEQ ID NO: 12) for PARP1.

[3] The composition of [2], wherein the double-stranded molecule, wherein the sense strand contains a sequence corresponding to a target sequence selected from among SEQ ID NOs: 5, 7, 8, 14 and 15.

[4] The composition of [1], wherein the cancer to be treated is pancreatic cancer and/or prostate cancer;

[5] The composition of [4], wherein the pancreatic cancer is pancreatic ductal adenocarcinoma (PDAC), and the prostate cancer is castration-resistant prostate cancer (CRPC);

[6] The composition of [1], wherein the composition contains plural kinds of the double-stranded molecules;

[7] The composition of [3], wherein the double-stranded molecule has a length of less than about 100 nucleotides;

[8] The composition of [7], wherein the double-stranded molecule has a length of less than about 75 nucleotides;

[9] The composition of [8], wherein the double-stranded molecule has a length of less than about 50 nucleotides;

[10] The composition of [9], wherein the double-stranded molecule has a length of less than about 25 nucleotides;

[11] The composition of [10], wherein the double-stranded molecule has a length of between about 19 and about 25 nucleotides;

[12] The composition of [1], wherein the double-stranded molecule is composed of a single polynucleotide containing the sense strand and the antisense strand linked by an intervening single-strand;

[13] The composition of [12], wherein the double-stranded molecule has the general formula 5′-[A]-[B]-[A′]-3′, wherein [A] is the sense strand sequence contains a sequence corresponding to a target sequence selected from among SEQ ID NOs: 5, 7, 8, 14 and 15, [B] is the intervening single-strand consisting of 3 to 23 nucleotides, and [A′] is the antisense strand contains a sequence complementary to [A];

[14] The composition of [1], wherein the double-stranded molecule is an RNA;

[15] The composition of [1], wherein the double-stranded molecule is DNA and/or RNA;

[16] The composition of [15], wherein the double-stranded molecule is a hybrid of a DNA polynucleotide and an RNA polynucleotide;

[17] The composition of [16], wherein the sense and antisense strand polynucleotides are composed of DNA and RNA, respectively;

[18] The composition of [15], wherein the double-stranded molecule is a chimera of DNA and RNA;

[19] The composition of [18], wherein a region flanking to the 3′-end of the antisense strand, or both of a region flanking to the 5′-end of sense strand and a region flanking to the 3′-end of antisense strand are composed of RNA;

[20] The composition of [19], wherein the flanking region is composed of 9 to 13 nucleotides;

[21] The composition of [1], wherein the double-stranded molecule contains 3′ overhangs;

[22] The composition of [1], wherein the composition includes a transfection-enhancing agent and pharmaceutically acceptable carrier.

[23] The composition of [1], wherein the double-stranded molecule is encoded by a vector and contained in the composition;

[24] The composition of [23], wherein the double-stranded molecule encoded by the vector acts at mRNA which matches a target sequence selected from among SEQ ID NO: 5 (at the position 595-613 nt of SEQ ID NO: 10), SEQ ID NO: 7 (at the position 1133-1151 nt of SEQ ID NO: 10), SEQ ID NO: 8 (at the position 1310-1328 nt of SEQ ID NO: 10) and SEQ ID NO: 14 (at the position 606-624 of SEQ ID NO: 10) for C12ORF48 gene, and SEQ ID NO: 15 (at the position 2685-2703 nt of SEQ ID NO: 12) for PARP1.

[25] The composition of [24], wherein the sense strand of the double-stranded molecule encoded by the vector contains the sequence corresponding to a target sequence selected from among SEQ ID NOs: 5, 7, 8, 14 and 15.

[26] The composition of [23], wherein the cancer to be treated is pancreatic cancer or prostate cancer;

[27] The composition of [26], wherein the pancreatic cancer is pancreatic ductal adenocarcinoma (PDAC), and the prostate cancer is castration-resistant prostate cancer (CRPC);

[28] The composition of [23], wherein plural kinds of the double-stranded molecules are administered;

[29] The composition of [25], wherein the double-stranded molecule encoded by the vector has a length of less than about 100 nucleotides;

[30] The composition of [29], wherein the double-stranded molecule encoded by the vector has a length of less than about 75 nucleotides;

[31] The composition of [30], wherein the double-stranded molecule encoded by the vector has a length of less than about 50 nucleotides;

[32] The composition of [31], wherein the double-stranded molecule encoded by the vector has a length of less than about 25 nucleotides;

[33] The composition of [32], wherein the double-stranded molecule encoded by the vector has a length of between about 19 and about 25 nucleotides in length;

[34] The composition of [23], wherein the double-stranded molecule encoded by the vector is composed of a single polynucleotide containing both the sense strand and the antisense strand linked by an intervening single-strand;

[35] The composition of [23], wherein the double-stranded molecule has the general formula 5′-[A]-[B]-[A′]-3′, wherein [A] is the sense strand containing a sequence corresponding to a target sequence selected from among SEQ ID NOs: SEQ ID NOs: 5, 7, 8, 14 and 15, [B] is a intervening single-strand composed of 3 to 23 nucleotides, and [A′] is the antisense strand containing a sequence complementary to [A]; and

[36] The composition of [23], wherein the composition includes a transfection-enhancing agent and pharmaceutically acceptable carrier.

Suitable compositions of the present invention are described in additional detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to the same target sequence, or different target sequences of C12ORF48 or PARP1. For example, the composition may contain double-stranded molecules directed to C12ORF48 or PARP1. Alternatively, for example, the composition may contain double-stranded molecules directed to one, two or more target sequences C12ORF48 or PARP1.

Furthermore, the present composition may contain a vector coding for one or plural double-stranded molecules. For example, the vector may encode one, two or several kinds of the present double-stranded molecules. Alternatively, the present composition may contain plural kinds of vectors, each of the vectors coding for a different double-stranded molecule.

Moreover, the present double-stranded molecules may be contained as liposomes in the present composition. See under the item of “Methods of treating cancer using the double-stranded molecule” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can include 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition may contain other pharmaceutically active ingredients so long as they do not inhibit the in vivo function of the double-stranded molecules of the present invention. For example, the composition may contain chemotherapeutic agents conventionally used for treating cancers.

In another embodiment, the present invention provides for the use of the double-stranded nucleic acid molecules of the present invention in manufacturing a pharmaceutical composition for treating a pancreatic cancer and prostate cancer characterized by the expression of C12ORF48. For example, the present invention relates to a use of double-stranded nucleic acid molecule inhibiting the expression of a C12ORF48 or PARP1 gene in a cell, which molecule includes a sense strand and an antisense strand complementary thereto, hybridized to each other to form the double-stranded nucleic acid molecule and targets to a sequence selected from among SEQ ID NOs: 5, 7, 8, 14 and 15, for manufacturing a pharmaceutical composition for treating pancreatic cancer and prostate cancer expressing C12ORF48.

The present invention further provides a method or process for manufacturing a pharmaceutical composition for treating a pancreatic cancer or prostate cancer characterized by the expression of C12ORF48, wherein the method or process includes a step for formulating a pharmaceutically or physiologically acceptable carrier with a double-stranded nucleic acid molecule inhibiting the expression of C12ORF48 or PARP1 in a cell, which over-expresses the gene, which molecule includes a sense strand and an antisense strand complementary thereto, hybridized to each other to form the double-stranded nucleic acid molecule and targets to a sequence selected from among SEQ ID NOs: 5, 7, 8, 14 and 15 as active ingredients.

In another embodiment, the present invention provides a method or process for manufacturing a pharmaceutical composition for treating a pancreatic cancer and prostate cancer characterized by the expression of C12ORF48, wherein the method or process includes a step for admixing an active ingredient with a pharmaceutically or physiologically acceptable carrier, wherein the active ingredient is a double-stranded nucleic acid molecule inhibiting the expression of C12ORF48 or PARP1 in a cell, which over-expresses the gene, which molecule includes a sense strand and an antisense strand complementary thereto, hybridized to each other to form the double-stranded nucleic acid molecule and targets to a sequence selected from among SEQ ID NOs: 5, 7, 8, 14 and 15.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### EXAMPLES

The invention will be further described in the following examples, which do not limit the scope of the invention described in the claims.

General Methods

1. Cell Lines.

PDAC cell lines KLM-1, SUIT-2, KP-1N, PK-1, PK-45P and PK-59 were provided from Cell Resource Center for Biomedical Research, Tohoku University (Sendai, Japan). MIAPaCa-2 and Panc-1 were purchased from the American Type Culture Collection (ATCC, Rockville, Md.). COS7 cell and PC cell lines LNCaP, 22Rv1, PC-3 and DU-145 were also purchased from the American Type Culture Collection (ATCC, Rockville, Md.), and LNCaP-derived CRPC cell line C4-2B was purchased from ViroMed Laboratories (Minnetonka, Minn.). KLM-1, SUIT-2, PK-1, PK-45P, PK-59 and Panc-1, were grown in RPMI1640 (Sigma-Aldrich, St. Louis, Mo.), and COS7, MIAPaCa-2, LNCaP, C4-2B, 22Rv1, PC-3, and DU-145 in Dulbecco's Modified Eagle's Medium (Sigma-Aldrich), all with 10% fetal bovine serum and 1% antibiotic/antimycotic solution (Sigma-Aldrich). Cells were maintained at 37 degrees C. in atmospheres of humidified air with 5% CO₂.

2. Semi-Quantitative RT-PCR.

Purification of cancer cells and normal epithelial cells from frozen PDAC or CAPC tissues was described previously (Nakamura T, et al., Oncogene 2004; 23:2385-400, Tamura K, et al., Cancer Res 2007; 67:5117-25). RNAs from the purified cancer cells and normal epithelial cells were subjected to two rounds of RNA amplification using T7-based in vitro transcription (Epicentre Technologies, Madison, Wis.). Total RNAs from human cancer cell lines were extracted using Trizol reagent (Invitrogen) according to the manufacturer's recommendations. Extracted RNAs were treated with DNase I (Roche Diagnostic, Mannheim, Germany) and reversely-transcribed to single-stranded cDNAs using oligo (dT) primer with Superscript II reverse transcriptase (Invitrogen). Appropriate dilutions of each single-stranded cDNA were prepared for subsequent PCR amplification by monitoring beta-actin (ACTB) as a quantitative control. The sets of primer sequences were

5′-TTGGCTTGACTCAGGATTTA-3′ (SEQ ID NO: 1) and

reverse 5′-ATGCTATCACCTCCCCTGTG-3′ (SEQ ID NO: 2) for ACTB,

5′-CTCAGCTGGGAAAGCTACAGAT-3′ (SEQ ID NO: 3) and

5′-CATGCCAGGTAGTTCTTCCATC-3′ (SEQ ID NO: 4) for C12ORF48. All reactions involved initial denaturation at 94 degrees C. for 2 min followed by 23 cycles (for ACTB), 28 cycles (for C12ORF48) at 94 degrees C. for 30 s, 58 degrees C. for 30 s, and 72 degrees C. for 1 min, on a GeneAmp PCR system 9700 (PE Applied Biosystems, Foster, Calif.).

3. Northern Blotting Analysis.

One micro g poly A+ RNAs from seven PDAC cell lines (KLM-1, PK-59, PK-45P, MIAPaCa-2, Panc-1, PK-1, and SUIT-2) and seven adult normal tissues (heart, lung, liver, kidney, brain, testis, and pancreas, from BD Bioscience, Palo Alto, Calif.) were blotted to the membrane. For prostate cancer, one micro g poly A+ RNAs from five PC cell lines (22Rv1, LNCaP, C4-2B, DU145, and PC-3) and six adult normal tissues (heart, lung, liver, kidney, brain, and prostate, from BD Bioscience, Palo Alto, Calif.) were blotted to the membrane. These northern-blot membranes and human MTN blot membrane (Multiple Tissue Northern blot, BD Bioscience) were hybridized for 16 hours with ³²P-labeled GABRP probe, which was labeled using Mega Label kit (GE Healthcare, Piscataway, N.J.). Probe cDNA of C12ORF48 was prepared as a 305-bp PCR product by using primers for C12ORF48 described above. Pre-hybridization, hybridization, and washing were performed according to the manufacture's instruction. The blots were autoradiographed at −80 degrees C. for 10 days.

4. Small Interfering RNA (siRNA)-Expressing Vectors Specific to C12ORF48.

To knock down endogenous C12ORF48 expression in PDAC cells, psiU6BX3.0 vector was used for expression of short hairpin RNA against a target gene as described previously (Tamura K, et al., Cancer Res 2007; 67:5117-25). The U6 promoter was cloned upstream of the gene-specific sequence (19-nt sequence from the target transcript, separated from the reverse complement of the same sequence by a short spacer, TTCAAGAGA), with five thymidines as a termination signal and a neo cassette for selection by Geneticin (Sigma-Aldrich). The target sequences for C12ORF48 were 5′-CACAGTATCTCCTAGTCAA-3′(#595) (SEQ ID NO: 5),

5′-CATGCTGCTCGAGAGAAAC-3′(#851) (SEQ ID NO: 6),

5′-GTTGCTCAGGATTTGGATT-3′(#1133) (SEQ ID NO: 7),

### 5′-GCAGCTAATGCTCCTACCA-3′(#1310) (SEQ ID NO: 8), and

5′-GAAGCAGCACGACTTCTTC-3′(siEGFP) (SEQ ID NO: 9) as a negative control. Human PDAC cell lines, PK-59 and MiaPaCa2, were plated on 10-cm dishes, and transfected with these siRNA-expression vectors using FuGENE6 (Roche) according to manufacturer's instruction, followed by 150 microg/ml (for PK-59) or 800 microg/ml (for MiaPaCa2) Geneticin (GIBCO) selection. The cells from 10-cm dishes were harvested 7 days later to analyze the knockdown effect on C12ORF48 by RT-PCR using the above primers. After cultured in appropriate medium containing Geneticin for 2 weeks, the cells were fixed with 100% methanol, stained with 0.1% of crystal violet-H₂O for colony formation assay. In MTT assay, cell viability was measured using Cell-counting kit-8 (DOJINDO, Kumamoto, Japan) at 6 days after the transfection. Absorbance was measured at 490 nm, and at 630 nm as reference, with a Microplate Reader 550 (Bio-Rad, Hercules, Calif.).

5. Immunocytochemistry.

COS7 cells were transfected with HA-tagged C12ORF48 expression vector by using Fugene (Roche) according to the manufacturer's recommended procedures, and 72 hours after the treatment, the cells were fixed with 4% paraformaldehyde, and permeablilized with 0.1% Triton X-100 in PBS for 1 min at room temperature. Non-specific binding was blocked by treatment with PBS containing 3% BSA for 30 min at room temperature. The cells were incubated for 60 min at room temperature with rabbit anti-HA antibody (3F10, Roche) diluted in PBS containing 1% BSA (1:1,000). After washing with PBS, the cells were stained by FITC-conjugated secondary antibody (Santa Cruz) for 60 min at room temperature. After washing with PBS, specimen was mounted with VECTASHIELD (VECTOR Laboratories, Inc, Burlingame, Calif.) containing 4′,6′-diamidine-2′-phenylindolendihydrochrolide (DAPI) and visualized with Spectral Confocal Scanning Systems (Leica, Bensheim, Germany).

6. Generation of Antibodies Specific to C12ORF48 Protein and Immunohistochemical staining.

Plasmids were designed to express two fragments of C12ORF48 (codons 1-150 and 328-498) using pET21a (+) vector in frame with a T7 tag at the N-terminus and a histidine (His) tag at the C-terminus (Novagen, Madison, Wis., USA), respectively. The two recombinant proteins were expressed in Escherichia coli, BL21 codon-plus strain (Stratagene, La Jolla, Calif., USA) and purified using Ni-NTA resin agarose (Qiagen, Valencia, Calif., USA) according to the supplier's protocols. The purified recombinant proteins were mixed together and then used for immunization of rabbits. The immune sera were subsequently purified on antigen affinity columns using Affigel 15 gel (Bio-Rad Laboratories, Hercules, Calif., USA) according to the supplier's instructions. Conventional tissue sections from PDACs were obtained from surgical specimens that were resected at the Osaka Medical Center for Cancer and Cardiovascular Diseases under the appropriate informed consent. The sections were deparaffinized and autoclaved at 108 degrees C. in citrate buffer pH 6.0 for 15 min. Endogenous peroxidase activity was quenched by incubation in Peroxidase Blocking Reagent (Dako Cytomation, Carpinteria, Calif., USA) for 30 min. After incubation in fetal bovine serum for blocking, the sections were incubated with rabbit anti-C12ORF48 polyclonal antibody (dilution 1:2500) at room temperature for 1 h. After washing with phosphate-buffered saline (PBS), immunodetection was performed with peroxidase labeled antirabbit immunoglobulin (Envision kit; Dako Cytomation). Finally, the reactants were developed with 3,3′-diaminobenzidin. Counterstaining was performed using hematoxylin.

7. Immunoprecipitation and Mass-Spectrometric Analysis for C12ORF48-Interacting Proteins.

The pCAGGSn3xFlag-C12ORF48—HA or empty pCAGGSn3FH mock were transfected into HEK293 cells using FuGENE6 (Roche). Forty-eight hours after the transfection, the cells were collected and lysed in lysis buffer (50 mmol/L Tris-HC1 [pH 8.0], 0.4% NP-40, 150 mmol/L NaCl, Protease Inhibitor Cocktail Set III [Calbiochem, San Diego, Calif., USA]). Cell extracts were precleared by incubation at 4 degrees C. for 1 h with 60 micro 1 of CL-4B sepharose (Sigma). After centrifugation, the supernatant was incubated at 4 degrees C. for 1 h with 30 micro 1 anti-FLAG M₂-agarose (Sigma). The beads were then collected by centrifugation at 8,000 rpm for 2 min and washed 5 times with 1 mL of immunoprecipitation buffer. The proteins were separated in 5% to 20% SDS-PAGE gels (Bio-Rad) and stained with a silver-staining kit. Protein bands that specifically found in the cell extracts transfected with C12ORF48 were excised and were analyzed by liquid chromatography-mass spectrometry (LC-MS/MS) analysis.

The excised bands were reduced in 10 mM tris(2-carboxyethyl)phosphine (Sigma) with 50 mM ammonium bicarbonate (Sigma) for 30 min at 37 degrees C. and alkylated in 50 mM iodoacetamide (Sigma) with 50 mM ammonium bicarbonate for 45 min in the dark at 25 degrees C. Porcine trypsin (Promega, San Luis Obispo, Calif.) was added for a final enzyme to protein ratio of 1:20. The digestion was conducted at 37 degrees C. for 16 hours. The resulting peptide mixture was separated on a 100 micro m×150 mm HiQ-Sil C18W-3 column (KYA Technologies, Tokyo, Japan) using 30 min linear gradient from 5.4 to 29.2% acetonitrile in 0.1% trifluoroacetic acid (TFA) with total flow of 300 nl/min. The eluting peptides were automatically mixed with matrix solution (4 mg/ml alpha-cyano-4-hydroxy-cinnamic acid (SIGMA), 0.08 mg/ml ammonium citrate in 70% acetonitrile, 0.1% TFA) and spotted onto MALDI target plates by MaP (KYA Technologies). Mass spectrometric analysis was performed on 4800 Plus MALDI/TOF/TOF Analyzer (Applied Biosystems/MDS Sciex). MS/MS peak list was generated by the Protein Pilot version 2.0.1 software (Applied Biosystems/MDS Sciex) and exported to a local MASCOT search engine version 2.2.03 (Matrix Science) for protein data base search.

To confirm the interaction between C12ORF48 and PARP1 proteins, FlagC12ORF48 expression vector and/or PARP1-Myc expression vector were cotransfected into HEK 293 cells. The transfected cells were lysed as described above and immunoprecipitated with c-Myc antibody (Santa Cruz). The co-precipitated proteins were immunoblotted using anti-Flag antibody (Sigma) and c-Myc antibody.

8. Flow Cytometry.

KLM-1 cells were respectively transfected with C12ORF48 siRNA duplex (5′-CUAGUCAACUACUGGAUUU-3′) (SEQ ID NO: 14), PARP1 siRNA duplex (5′-GAUAGAGCGUGAAGGCGAA-3′) (SEQ ID NO: 15), and siEGFP duplex (5′-GAAGCAGCACGACUUCUUC-3′) (SEQ ID NO: 9) as a negative control. Seventy-two hours after the treatment, the cells were trypsinized and collected, fixed with 70% ethanol in PBS at 4 degrees C., rinsed twice in PBS, and incubated at 37 degrees C. for 30 min with 500 micro 1 of PBS containing 0.5 mg of boiled RNase. The cells were stained in 500 micro 1 of PBS containing 25 micro g of propidium iodide. The percentages of sub-G1 nuclei (apoptotic cells) in each population were determined from at least 2×10⁴ cells by means of a flow cytometer (Beckman Coulter).

9. In-Vitro PARP-1 auto-poly(ADP-ribosyl)ation Assays.

In-vitro PARP1 automodification assays were performed as described previously (10). Briefly, 200 ng of the purified C12ORF48 recombinant protein and 25 ng of the recombinant human PARP1 (Alexis) were incubated for 10 min at 37 degrees C. in binding buffer (10 mM Tris-HCl, pH 7.5, 1 mM MgCl₂, 1 mM DTT) plus 10 micro g/ml of sonicated DNA. The reactions were started by adding 5 micro Ci (0.25 micro M) ³²P-labeled NAD+, and incubated at 37 degree for 10 additional minutes. After terminating the reactions with SDS sample buffer, the proteins were fractionated by 8% SDS-PAGE gel. Poly (ADP-ribosyl)ated proteins were visualized by autoradiography.

10. PARP Activity in Cell Extracts.

KLM-1 and SUIT-2 were transfected with C12ORF48-siRNA, PARP1-siRNA, or siEGFP (as a control) and collected 72 h after transfection. Western blot analysis using anti-C12ORF48 antibody and anti-PARP1 antibody (TREVIGEN, Gaithersburg, Md.) confirmed the knockdown effect their expression on KLM-1 and SUIT-2 cells. PARP1 activities in cell extracts were assayed using the universal colorimetric PARP assay kit (TREVIGEN) based on the incorporation of biotinylated ADP-ribose onto histone H1 proteins. Cell extracts were loaded into a 96-well plate coated with histones and biotinylated poly ADP-ribose, allowed to incubate for 1 h, treated with strep-HRP, and read at 450 nm in a spectrometrophotometer. PARP1 enzymatic activities were also confirmed by using anti-poly (ADP-ribose) (PAR) mouse monoclonal affinity purified antibody (TREVIGEN). 25 micro g cell extracts or 5 ng recombinant human PARP1 (TREVIGEN) were incubated for 20 min at 37 degrees C. in binding buffer (10 mM TrisHCl, pH 7.5, 1 mM MgCl₂, 1 mM DTT) plus 10 micro g/ml of sonicated DNA, and 200 micro M NAD+ (Sigma).

Results

Over-Expression of C12ORF48 in PDAC Cells and PC Cells.

Among dozens of trans-activated genes that were screened by our genome-wide cDNA microarray analysis of PDAC cells (Nakamura T, et al., Oncogene 2004; 23:2385-400) and CRPC cells (Tamura K, et al., Cancer Res 2007; 67:5117-25), the present inventors focused on C12ORF48. C12ORF48 over-expression was confirmed by RT-PCR in five of the nine microdissected-PDAC cell populations (FIG. 1A) and two of five microdissected-CRPC cell populations (FIG. 1B). Northern-blot analysis using a C12ORF48 cDNA fragment as the probe identified an about 4-kb transcript only in the testis, but no expression was observed in any other organs including lung, heart, liver, kidney, and brain (FIG. 1C). C12ORF48 expression in several PDCA cell lines (FIG. 1D) and prostate cancer cell line (FIG. 1E) were also examined and expression thereof was evidently found in many PDAC or prostate cancer cell lines examined.

Effect of C12ORF48-siRNA on Growth of Cancer Cells.

To investigate the biological significance of C12ORF48 expression in cancer cells, four siRNA-expression vectors specific to C12ORF48 transcript were constructed and transfected into PDAC cell lines, MiaPaCa2 or PK-59 cells that endogenously expressed high levels of C12ORF48. Knockdown effect was observed by RT-PCR when si#595 (target sequence: SEQ ID NO: 5), si#1133 (target sequence: SEQ ID NO: 7), and si#1310 (target sequence: SEQ ID NO: 8) were transfected, but not in the case of si#851 (target sequence: SEQ ID NO: 6) or a negative control siEGFP (target sequence: SEQ ID NO: 9) (FIG. 2A left). Colony-formation and MTT assays (FIG. 2B, 2C left) using MiaPaCa2 revealed a drastic reduction in the number of cells transfected with si#595, si#1133, and si#1310, compared with si#851 and siEGFP for which no knockdown effect was observed. The similar results were obtained when these siRNA-expression vectors were transfected into PK-59 cells (FIGS. 2A, B and C, right).

C12ORF48 Protein was Localized in the Nucleus.

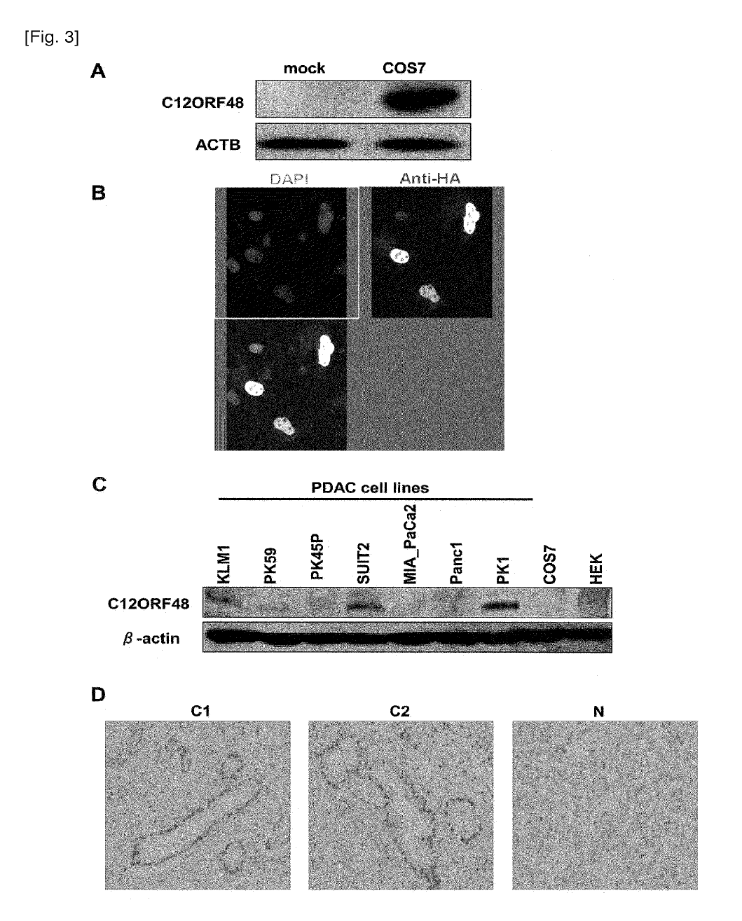

C12ORF48 protein had any significant motif or domain predicted, but it was predicated to be localized in the nucleus by in silico analysis. To confirm its intracellular localization, C12ORF48-expression vector was transfected into COS7 cells (FIG. 3A) and performed immunocytochemical analysis using anti-HA tag antibody. As shown in FIG. 3B, immunocytochemical analysis clearly showed that exogenous C12ORF48 protein was localized in the nucleus, which suggested its involvement with the chromatin or DNA structure. To examine its expression at the protein level, we generated a polyclonal antibody specific to C12ORF48 protein, and performed western blot analysis that detected endogenous C12ORF48 in most of all the PDAC cell lines we examined (FIG. 3C). C12ORF48 expression in PDAC cell lines was higher than that in non-cancerous cell lines (HEK-293, and COS7). Immunohistochemical analysis were also performed using clinical PDAC tissue sections and found its strong positive staining in the nuclei of PDAC cells (Panels C₁-C₂ in FIG. 3D), while, no or very limited staining was detected in normal pancreas tissue (N in FIG. 3D). Collectively, 33 out of 62 PDAC tissues (53%) revealed positive staining for C12ORF48.

Identification of PARP1 as an Interacting Protein of C12ORF48.

Since the biological functions of C12ORF48 are totally unknown, a protein(s) interacting with C12ORF48 were searched by immunoprecipitation and mass spectrometry analyses. Lysates of HEK293 cells transfected with C12ORF48-Flag-expression vector or an empty vector (mock control) were extracted and immunoprecipitated with anti-Flag M₂-agarose. Immunoprecipitaed protein complexes were silver-stained on SDS-PAGE gel. An approximately 110 kD protein was observed in the co-immunoprecipitates of cell lysates transfected with Flag-tagged C12ORF48 plasmid, but not in those with mock control plasmid (FIG. 4A), and we extracted this 110 kD band for LC-MS/MS analysis. Mass-spectrometric analysis identified PARP1 as a candidate protein interacting with C12ORF48. This result was confirmed by western blot analysis using anti-PARP1 antibody (FIG. 4B). Furthermore, to confirm their physical interaction, Myc-tagged PARP1 expression vector and/or Flag-tagged C12ORF48 expression vector into HECK293, and conversely, immunoprecipitation were performed using anti-Myc antibody and then immunoblotted the precipitates using anti-Flag antibody. The results showed that C12ORF48 was co-precipitated with PARP1 (FIG. 4C).

Induction of Apoptosis by Oligo C12ORF48— siRNA Duplex or Oligo PARP1-siRNA Duplex.

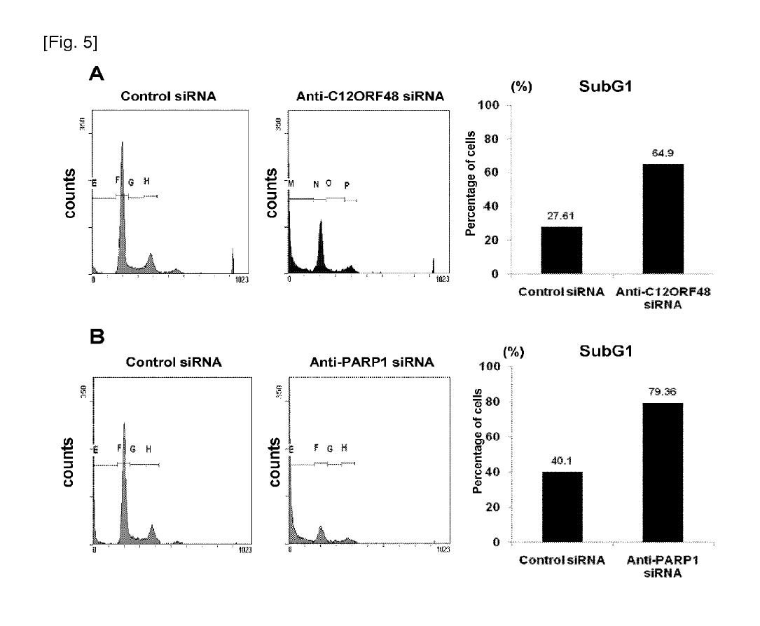

FACS analysis detected a larger proportion of subG1 populations in KLM-1 cells respectively transfected with C12ORF48-siRNA (FIG. 5A), or PARP1-siRNA (FIG. 5B) compared with cells transfected with control siRNA. Similar results were also observed when they were knocked down in other PDAC SUIT-2 cells (data not shown).

C12ORF48 could Positively Regulate PARP1 Automodification In Vitro.

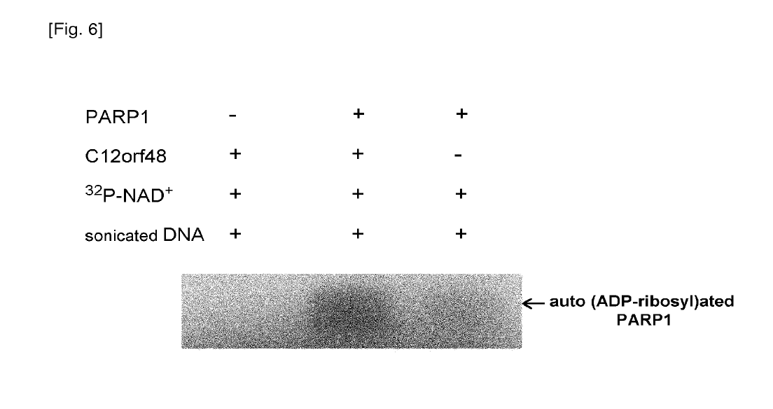

The major protein that PARP1 could poly(ADP-ribosyl)ate is PARP1 itself. To investigate the functional consequences of the interaction between PARP1 and C12ORF48, PARP1 automodification was measured by incorporation of [³²P] NAD+ and visualized by SDS-polyacrylamide gel electrophoresis in the absence or in the presence of purified C12ORF48 protein. As shown in FIG. 6, PARP1 automodification was strongly enhanced in the presence of C12ORF48 protein compared with in the absence of C12ORF48 protein.

C12ORF48 could Regulate PARP1 Activity in Cancer Cell Extracts.

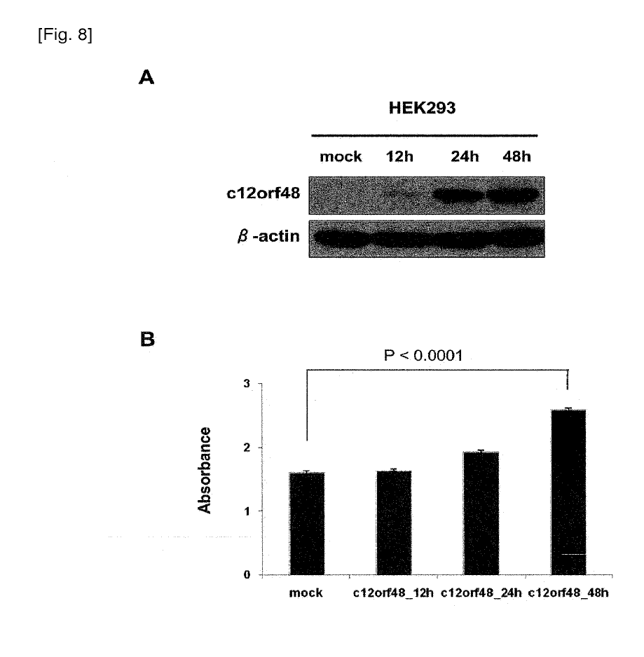

To investigate for the functional significance of C12ORF48 to PARP1 activity in cancer cells, the PARP1 activities were examined in cell extracts transfected with C12ORF48-siRNA, PARP1-siRNA, and control siRNA, by using the colorimetric PARP assay based on the incorporation of biotinylated ADP-ribose onto histone H1 proteins. First, it was validated that transfection of siRNA duplex to C12ORF48 or PARP1 into KLM-1 cells decreased their protein expressions (FIG. 7A). The colorimetric PARP assay found the PARP1 activities to poly(ADP-ribosyl)ate histone H1 in KLM-1 cell extracts transfected with C12ORF48-siRNA, or PARP1-siRNA were decreased 59.2%, and 55.5% respectively, compared with control-siRNA (FIG. 7B). It was also observed that the PARP1 activities were decreased 65.2% and 47.1% in other PDAC Cell line SUIT-2 cells in knockdown of C12ORF48 or PARP1, respectively (FIG. 7C, D). Furthermore, western blot analysis using anti-poly(ADP-ribose) (PAR) antibody also confirmed that PARP1 activity was drastically decreased in the cell extracts in C12ORF48 or PARP1 knockdown (FIG. 7E). These findings indicated that C12ORF48 could regulate poly-(ADP-ribosyl)ation activity of PARP1 to histone H1 and other target protein including PARP1 itself. Next, C12ORF48 was over-expressed in HEK293 cells and PARP activity was examined by the colorimetric PARP assay, as well. Exogenous C12ORF48 expression was induced in time-dependent manner (FIG. 8A), and concordantly with C12ORF48 expression, the PARP1 activity in HECK293 cell extracts were also enhanced (FIG. 7B, P<0.0001, vs mock transfection) in time-dependent manner. Taken together, our findings suggest that C12ORF48 could positively regulate PARP1 enzyme activity both in vivo and in vitro.

Discussion

In the present invention, one novel target gene, C12ORF48, was identified through microarray analysis as over-expressed in PDAC cells and CRPC cells and its over-expression was validated in PDAC cells and CRPC cells by RT-PCR. Since it was restrictively expressed in the testis among the adult normal organs, C12ORF48 appears to be an appropriate and promising molecular target for a novel therapeutic approach with minimal adverse effect. Furthermore, functional knockdown of endogenous C12ORF48 by siRNA in pancreas cancer cell lines resulted in drastic suppression of pancreatic cancer cell growth, suggesting its essential role in maintaining viability of cancer cells.

The present inventers found C12ORF48 can interact with poly(ADP-ribose) polymerase-1 (PARP1). PARP1, the most abundant nuclear protein after histones, possesses an intrinsic enzymatic activity that catalyzes the transfer of ADP-ribose units from donor NAD+ molecules to target proteins as monomers, oligomers, or polymers of ADP-ribose. PARP1 mediates chromatin loosening and activates the transcription of inducible genes. To date, it is well known that PARP1 plays critical roles in DNA repair, cell death pathways, chromatin remodeling, and so on. However, considerably less is known about the chromatin-dependent gene regulatory activities of PARP1 under physiological conditions where the integrity of the genome is maintained.

These results also collectively suggested that both C12ORF48 and PARP1 were nuclear proteins. C12ORF48 could positively regulate PARP1 automodification in vitro. In addition, suppression of C12ORF48 protein could reduce the activity of PARP1 in cancer cell extracts, not only the activities to poly (ADP-ribosyl)ate histone

H1, but to other targets include PARP1 itself. Furthermore, the inventers also investigated overexpression of C12ORF48 could enhance the activity of PARP1 in cell extracts. All of these data implied that C12ORF48 could be involved in DNA repair, transcriptional regulation, chromatin modification, and cell signaling through the regulation of PARP1 activities.

### INDUSTRIAL APPLICABILITY

The gene-expression analysis of cancers described herein using the combination of laser-capture dissection and genome-wide cDNA microarray has identified C12ORF48 as a target gene for cancer prevention and therapy. Based on its differential expression, the present invention confirms the utility of C12ORF48 as a molecular diagnostic marker for identifying and detecting cancer, in particular, pancreatic and prostate cancer.

The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provides indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of detecting or diagnosing cancer in a subject, comprising determining an expression level of a C12ORF48 gene in a subject-derived biological sample, wherein an increase of said level as compared to a normal control level of the gene indicates that the subject suffers from or is at risk of developing cancer, wherein the expression level is determined by any one method selected from the group consisting of:
(a) detecting mRNA of the C12ORF48 gene,
(b) detecting a protein encoded by the C12ORF48 gene, and
(c) detecting a biological activity of a protein encoded by the C12ORF48 gene.

2. The method of claim 1, wherein said increase is at least 10% greater than said normal control level.

3. The method of claim 1, wherein the subject-derived biological sample is biopsy.

4. The method of claim 1, wherein the cancer is selected from the group of pancreatic cancer and prostate cancer.

5. The method of claim 4, wherein the pancreatic cancer is pancreatic ductal adenocarcinoma, and the prostate cancer is castration-resistant prostate cancer.

6. A kit for diagnosing cancer, which comprises a reagent selected from the group consisting of:
(a) a reagent for detecting mRNA of a C12ORF48 gene;
(b) a reagent for detecting a protein encoded by a C12ORF48 gene; and
(c) a reagent for detecting a biological activity of a protein encoded by a C12ORF48 gene.

7. The kit of claim 6, wherein the reagent is a probe to a gene transcript of the gene.

8. The kit of claim 6, wherein the reagent is an antibody against the protein encoded by the gene.

9. A method of screening for a candidate compound for treating or preventing a cancer associated with the over-expression of a C12ORF48 gene or inhibiting cancer cell growth, the method comprising the steps of:
a) contacting a test compound with a polypeptide encoded by the C12ORF48 gene;
b) detecting a biological activity of the polypeptide of step (a) or detecting the binding activity between the polypeptide and the test compound; and
c) selecting a compound that suppresses the biological activity of the polypeptide in comparison with the biological activity detected in the absence of the test compound or selecting a compound that binds to the polypeptide.

10. A method of screening for a candidate compound for treating or preventing a cancer associated with the over-expression of a C12ORF48 or PARP1 gene or inhibiting cancer cell growth, the method comprising the steps of
a) contacting a test compound with a cell expressing a C12ORF48 gene; and
b) selecting a compound that reduces the expression level of the C12ORF48 gene.

11. (canceled)

12. The method of claim 9, wherein the biological activity is cell proliferative activity.

13. A method of screening for a candidate compound for treating or preventing a cancer associated with the over-expression of a C12ORF48 gene or inhibiting cancer cell growth, the method comprising the steps of:
a) contacting a test compound with a cell into which a vector comprising the transcriptional regulatory region of the C12ORF48 gene and a reporter gene that is expressed under the control of the transcriptional regulatory region has been introduced;
b) measuring the expression or activity of said reporter gene; and
c) selecting a compound that reduces the expression or activity level of said reporter gene, as compared to a level in the absence of the test compound.

14. The method of claim 9, wherein the cancer is selected from the group of pancreatic cancer and prostate cancer.

15. The method of claim 14, wherein the pancreatic cancer is pancreatic ductal adenocarcinoma, and the prostate cancer is castration-resistant prostate cancer.

16. A double-stranded molecule comprising a sense strand and an antisense strand, wherein the sense strand comprises a nucleotide sequence corresponding to a target sequence consisting of SEQ ID NO: 5, 7, 8, 14 or 15, and wherein the antisense strand comprises a nucleotide sequence which is complementary to the sense strand, wherein the sense strand and the antisense strand hybridize to each other to form the double-stranded molecule, and wherein the double-stranded molecule, when introduced into a cell expressing the C12ORF48 gene, inhibits expression of the gene.

17. The double-stranded molecule of claim 16, wherein the double-stranded molecule is an oligonucleotide of between about 19 and about 25 nucleotides in length.

18. The double-stranded molecule of claim 17, wherein the double-stranded molecule is a single nucleotide transcript comprising the sense strand and the antisense strand linked via a single-stranded nucleotide sequence.

19. The double-stranded molecule of claim 18, wherein the polynucleotide has the general formula 5′-[A]-[B]-[A′]-3′ wherein [A] is a nucleotide sequence comprising SEQ ID NO: 5, 7, 8, 14 or 15; [B] is a nucleotide sequence consisting of about 3 to about 23 nucleotides; and [A′] is a nucleotide sequence complementary to [A].

20. A vector comprising each or both of a combination of polynucleotide comprising a sense strand nucleic acid and an antisense strand nucleic acid, wherein the sense strand nucleic acid comprises a nucleotide sequence of SEQ ID NO: 5, 7, 8, 14 or 15, and wherein the antisense strand comprises a nucleotide sequence which is complementary to the sense strand, wherein the transcripts of the sense strand and the antisense strand hybridize to each other to form the double-stranded molecule, and wherein the vector, when introduced into a cell expressing the C12ORF48 gene, inhibits expression of the gene.

21. The vector of claim 20, wherein the polynucleotide is an oligonucleotide of between about 19 and about 25 nucleotides in length.

22. The vector of claim 20, wherein the double-stranded molecule is a single nucleotide transcript comprising the sense strand and the antisense strand linked via a single-stranded nucleotide sequence.

23. The vector of claim 22, wherein the polynucleotide has the general formula 5′-[A]-[B]-[A′]-3′ wherein [A] is a nucleotide sequence comprising SEQ ID NO: 5, 7, 8, 14 or 15; [B] is a nucleotide sequence consisting of about 3 to about 23 nucleotides; and [A′] is a nucleotide sequence complementary to [A].

24. A method of treating or preventing a cancer associated with the over-expression of a C12ORF48 gene in a subject comprising administering to the subject a pharmaceutically effective amount of a double-stranded molecule against C12ORF48 or a vector comprising the double-stranded molecule that inhibits the cell proliferation when said double-stranded molecule is introduced into a cell expressing C12ORF48 gene, and a pharmaceutically acceptable carrier.

25. The method of claim 24, wherein the double stranded molecule comprises a sense strand and an antisense strand, wherein the sense strand comprises a nucleotide sequence corresponding to a target sequence consisting of SEQ ID NO: 5, 7, 8, 14 or 15, and wherein the antisense strand comprises a nucleotide sequence which is complementary to the sense strand, wherein the sense strand and the antisense strand hybridize to each other to form the double-stranded molecule, and wherein the double-stranded molecule, when introduced into a cell expressing the C12ORF48 gene, inhibits expression of the gene, and wherein the vector comprises each or both of a combination of polynucleotide comprising a sense strand nucleic acid and an antisense strand nucleic acid, wherein the sense strand nucleic acid comprises a nucleotide sequence of SEQ ID NO: 5, 7, 8, 14 or 15, and wherein the antisense strand comprises a nucleotide sequence which is complementary to the sense strand, wherein the transcripts of the sense strand and the antisense strand hybridize to each other to form the double-stranded molecule, and wherein the vector, when introduced into a cell expressing the C12ORF48 gene, inhibits expression of the gene.

26. The method of claim 24, wherein the cancer is selected from the group of pancreatic cancer and prostate cancer.

27. The method of claim 26, wherein the pancreatic cancer is pancreatic ductal adenocarcinoma, and the prostate cancer is castration-resistant prostate cancer.

28. A composition for treating or preventing a cancer associated with the over-expression of a C12ORF48 gene, which comprises a pharmaceutically effective amount of a double-stranded molecule against C12ORF48 or a vector comprising said double-stranded molecule that inhibits the cell proliferation when said double-stranded molecule is introduced into a cell expressing C12ORF48 gene, and a pharmaceutically acceptable carrier.

29. The composition of claim 28, wherein the double stranded molecule comprises a sense strand and an antisense strand, wherein the sense strand comprises a nucleotide sequence corresponding to a target sequence consisting of SEQ ID NO: 5, 7, 8, 14 or 15, and wherein the antisense strand comprises a nucleotide sequence which is complementary to the sense strand, wherein the sense strand and the antisense strand hybridize to each other to form the double-stranded molecule, and wherein the double-stranded molecule, when introduced into a cell expressing the C12ORF48 gene, inhibits expression of the gene, and wherein the vector comprises each or both of a combination of polynucleotide comprising a sense strand nucleic acid and an antisense strand nucleic acid, wherein the sense strand nucleic acid comprises a nucleotide sequence of SEQ ID NO: 5, 7, 8, 14 or 15, and wherein the antisense strand comprises a nucleotide sequence which is complementary to the sense strand, wherein the transcripts of the sense strand and the antisense strand hybridize to each other to form the double-stranded molecule, and wherein the vector, when introduced into a cell expressing the C12ORF48 gene, inhibits expression of the gene.

30. The composition of claim 28, wherein the cancer is selected from the group of pancreatic cancer and prostate cancer.

31. The composition of claim 30, wherein the pancreatic cancer is pancreatic ductal adenocarcinoma, and the prostate cancer is castration-resistant prostate cancer.

32. A method of screening for a candidate compound that inhibits a binding between a C12ORF48 polypeptide and a PARP1 polypeptide, the method comprising the steps of
(a) contacting a C12ORF48 polypeptide or functional equivalent thereof with a PARP1 polypeptide or functional equivalent thereof in presence of a test agent;
(b) detecting a binding between the polypeptides;
(c) comparing binding level detected in step (b) with those detected in absence of the test agent; and
(d) selecting the test agent that reduces or inhibits binding level compared with that detected in absence of the test agent in step (c).

33. The method of claim 32, wherein the functional equivalent of C12ORF48 comprises PARP1-binding domain.

34. A method of screening for a compound for treating or preventing cancer using the polypeptide encoded by a C12ORF48 gene including the steps of:
(a) contacting a test compound with a polypeptide encoded by a polynucleotide of C12ORF48 in the presence of a polypeptide encoded by a polynucleotide PARP1;
(b) detecting the biological activity of the polypeptide encoded by a polynucleotide of PARP1; and
(c) selecting the test compound that suppresses the biological activity of the polypeptide encoded by the polynucleotide of PARP1 as compared to the biological activity of the polypeptide detected in the absence of the test compound.

35. The method of claim 34, wherein the biological activity is auto modification activity.

36. The method of claim 10, wherein the cancer is selected from the group of pancreatic cancer and prostate cancer.

37. The method of claim 36, wherein the pancreatic cancer is pancreatic ductal adenocarcinoma, and the prostate cancer is castration-resistant prostate cancer.

38. The method of claim 13, wherein the cancer is selected from the group of pancreatic cancer and prostate cancer.

39. The method of claim 38, wherein the pancreatic cancer is pancreatic ductal adenocarcinoma, and the prostate cancer is castration-resistant prostate cancer.
